(19) United States
(12) Patent Application Publication
(10) Pub. No.: US 2005/0059658 A1
(43) Pub. Date: Mar. 17, 2005

(54) Pyridazinone

(76) Inventors: Tobias Wunberg, Solingen (DE); Ulrich Betz, Wuppertal (DE); Sabine Hallenberger, Wuppertal (DE); Gerald Kleymann, Salzuflen (DE); Thomas Lampe, Dusseldorf (DE); Susanne Nikolic, Monheim (DE); Jurgen Reefschlager, Oldenburg (DE); Franz Zumpe, Wuppertal (DE); Marcus Bauser, Wuppertal (DE); Wolfgang Bender, Wuppertal (DE); Rolf Grosser, Leverkusen (DE); Kerstin Henninger, Wuppertal (DE); Guy Hewlett, Wuppertal (DE); Axel Jensen, Velbert (DE); Jorg Keldenich, Wuppertal (DE); Holger Zimmermann, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/482,520

(22) PCT Filed: Jun. 17, 2002
(86) PCT No.: PCT/EP02/06641

(30) Foreign Application Priority Data
Jun. 28, 2001 (DE) 101 31 133.8

(51) Int. Cl. A61K 31/50

(52) U.S. Cl. 514/228.2; 514/247; 544/239; 544/63

(57) ABSTRACT

The invention relates to novel compounds, to a method for the production of said compounds and to the use thereof as medicaments, more particularly as antiviral agents, especially against cytomegalovirus.

The present invention relates to novel compounds, to processes for their preparation and to their use as medicaments, in particular as antiviral agents, in particular against cytomegaloviruses.

EP-A-071 102 describes benzotriazole-substituted pyridazinones for cardiovascular disorders. EP-A-839 1 describes benzimidazole-substituted pyridazinones for cardiovascular disorders and with antiviral action.

The present invention relates to compounds of the general formula (I) 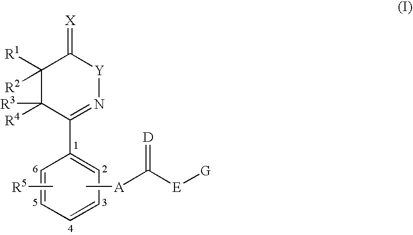
in which
A is attached via position 2, 3, 5 or 6 to the aromatic ring and
A represents oxygen or NR⁶,
E represents oxygen, CR⁹R¹⁰ or NR⁷,
Y represents oxygen or NR⁸,
D and X are identical or different and represent in each case oxygen or sulfur,
G represents hydrogen,
or
G represents C₆-C₁₀-aryl, where C₆-C₁₀-aryl may optionally be substituted by up to three substituents selected from the group consisting of halogen, hydroxyl, nitro, cyano, C₁-C₆-alkoxy, hydroxy-carbonyl, C₁-C₆-alkoxycarbonyl, amino, mono- or di-C₁-C₆-alkylamino, mono- or di-C₁-C₆-alkylaminocarbonyl and C₁-C₆-alkyl,
   where
   C₁-C₆-alkoxy, C₁-C₆-alkoxycarbonyl, mono- or di-C₁-C₆-alkylamino, mono- or di-C₁-C₆-alkylaminocarbonyl or C₁-C₆-alkyl may optionally be substituted by up to three substituents selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or C₁-C₆-alkylaminocarbonyl and C₆-C₁₀-aryl,
or
G represents C₆-C₁₀-aryl, where C₆-C₁₀-aryl may optionally be substituted by phenyl,
   where
   phenyl may optionally be substituted by up to three substituents selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₁-C₆-alkyl,
   where
   C₁-C₆-alkyl for its part may optionally be substituted by up to three substituents selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by phenyl,
   where
   phenyl may optionally be substituted by C₅-C₆-heteroaryl or C₅-C₇-heterocyclyl,
   where
   C₅-C₆-heteroaryl or C₅-C₇-heterocyclyl for their part may optionally be substituted by up to three substituents selected from the group consisting of halogen, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by a group of the following formula 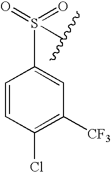
or
G represents C₅-C₁₀-heteroaryl or C₅-C₇-heterocyclyl, where C₅-C₁₀-heteroaryl or C₅-C₇-heterocyclyl may optionally be substituted by up to three substituents selected from the group consisting of halogen, nitro, cyano, C₁-C₆-alkyl, C₁-C₆-alkoxy, amino, mono- or di-C₁-C₆-alkylamino, hydroxycarbonyl, C₁-C₆-alkoxycarbonyl and mono or di-C₁-C₆-alkylaminocarbonyl,
or
G represents C₃-C₁₀-cycloalkyl, where C₃-C₁₀-cycloalkyl may optionally be substituted by up to three substituents selected from the group consisting of halogen, nitro, cyano, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono or di-C₁-C₆-alkylaminocarbonyl,
R¹, R², R³ and R⁴ are identical or different and each represent hydrogen, amino, mono- or di-C₁-C₆-alkylamino, C₁-C₆-alkylcarbonylamino, C₆-C₁₀-aryl or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono or di-C₁-C₆-alkylaminocarbonyl,
   and
   where C₆-C₁₀-aryl may optionally be substituted by up to three substituents selected from the group consisting of halogen, hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl, mono or di-C₁-C₆-alkylaminocarbonyl and C₁-C₆-alkyl,
   where
   C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R² or R³ and R⁴ together with the carbon atom to which they are attached form a C₃-C₆-cycloalkyl ring, where the C₃-C₆-cycloalkyl ring may optionally be substituted by up to three substituents selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R³ together with the carbon atoms to which they are attached form a C₃-C₆-cycloalkyl ring, where the C₃-C₆-cycloalkyl ring may optionally be substituted by up to three substituents selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁵ represents hydrogen, halogen, hydroxyl, C₁-C₆-alkoxy, amino, mono- or di-C₁-C₆-alkylamino or C₁-C₆-alkyl, where C₁-C₆-alkoxy, mono- or di-C₁-C₆-alkylamino or C₁-C₆-alkyl may optionally be substituted by up to three substituents selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁹ and R¹⁰ are identical or different and represent in each case hydrogen, NR¹¹R¹², OR¹³ or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹¹, R¹² and R¹³ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and their tautomers, stereioisomers, stereoisomeric mixtures and their pharmacologically acceptable salts.

In the context of the invention, C₁-C₃-alkyl, C₁-C₄-alkyl, C₁-C₆-alkyl represent a straight-chain or branched alkyl radical having 1 to 3, 1 to 4 and 1 to 6 carbon atoms, respectively. Examples which may be mentioned are: methyl, ethyl, n-propyl, isopropyl, n-butyl, isobutyl, t-butyl, n-pentyl and n-hexyl.

In the context of the invention, C₃-C₆-cycloalkyl, C₃-C₁₀-cycloalkyl represent a cycloalkyl group having 3 to 6 and 3 to 10 carbon atoms, respectively. Examples which may be mentioned are: cyclopropyl, cyclobutyl, cyclopentyl, cyclohexyl, cycloheptyl and adamantyl.

In the context of the invention, C₁-C₆-alkoxy represents a straight-chain, branched or cyclic alkyl radical having 1 to 6 carbon atoms which is attached via an oxygen atom. Examples which may be mentioned are: methoxy, ethoxy, n-propoxy, isopropoxy, t-butoxy, n-pentoxy and n-hexoxy. Unless indicated otherwise, preference is given to straight-chain or branched alkyl radicals having 1 to 6 carbon atoms, for example methoxy and ethoxy.

In the context of the invention, C₁-C₆-alkoxycarbonyl represents a straight-chain, branched or cyclic alkoxy radical having 1 to 6 carbon atoms which is attached via a carbonyl group. Examples which may be mentioned are: methoxycarbonyl, ethoxy-carbonyl, n-propoxycarbonyl, isopropoxycarbonyl and tert-butoxycarbonyl. Unless indicated otherwise, preference is given to straight-chain or branched alkoxy radicals having 1 to 6 carbon atoms.

In the context of the invention, C₆-C₁₀-aryl represents an aromatic radical having 6 to 10 carbon atoms. Preferred aryl radicals are phenyl and naphthyl.

In the context of the invention, mono-C₁-C₆-alkylamino represents an amino group having a straight-chain, branched or cyclic alkyl substituent which has 1 to 6 carbon atoms. Examples which may be mentioned are: methylamino, ethylamino, n-propylamino, isopropylamino, cyclopropylamino, t-butylamino, n-pentylamino, cyclopentylamino and n-hexylamino.

In the context of the invention, di-C₁-C₆-alkylamino represents an amino group having two identical or different straight-chain, branched or cyclic alkyl substituents, each of which has 1 to 6 carbon atoms. Examples which may be mentioned are: N,N-dimethylamino, N,N-diethylamino, N-ethyl-N-methylamino, N-methyl-N-n-propylamino, N-methyl-N-cyclopropylamino, N-isopropyl-N-n-propylamino, N-t-butyl-N-methylamino, N-ethyl-N-n-pentylamino and N-n-hexyl-N-methylamino.

In the context of the invention, mono-C₁-C₆-alkylaminocarbonyl represents an amino group having a straight-chain, branched or cyclic alkyl substituent which has 1 to 6 carbon atoms and which is attached via a carbonyl group. Examples which may be mentioned are: methylaminocarbonyl, ethylaminocarbonyl, n-propylaminocarbonyl, isopropylaminocarbonyl, cyclopropylaminocarbonyl, t-butylaminocarbonyl, n-pentylaminocarbonyl, cyclopentylaminocarbonyl and n-hexylaminocarbonyl.

In the context of the invention, di-C₁-C₆-alkylaminocarbonyl represents an amino group having two identical or different straight-chain, branched or cyclic alkyl substituents, each of which has 1 to 6 carbon atoms, and which is attached via a carbonyl group. Examples which may be mentioned are: N,N-dimethyl-aminocarbonyl, N,N-diethylaminocarbonyl, N-ethyl-N-methylaminocarbonyl, N-methyl-N-n-propylaminocarbonyl, N-methyl-N-cyclopropylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl.

In the context of the invention, halogen generally represents fluorine, chlorine, bromine and iodine. Preference is given to fluorine, chlorine and bromine. Particular preference is given to fluorine and chlorine.

In the context of the invention, 5- to 10-membered heteroaryl (“C₅-C₁₀-heteroaryl”) represents 5- to 10-membered aromatic rings which comprise heteroatoms and have at least one aromatic ring and which may comprise 1 to 4 heteroatoms selected from the group consisting of O, S and N. Heteroaryl for its part may also be substituted via C or N. Examples which may be mentioned are: pyridyl, furyl, thienyl, pyrrolyl, imidazolyl, pyrazolyl, pyrazinyl, pyrimidinyl, pyridazinyl, indolicenyl, indolyl, benzo[b]thienyl, benzo[b]furyl, indazolyl, quinolyl, isoquinolyl, naphthyridinyl, quinazolinyl, etc.

In the context of the invention, a 5- to 7-membered saturated or partially unsaturated heterocycle (“C₅-C₇-heterocyclyl”) having up to 3 heteroatoms from the group consisting of S, N and O generally represents a heterocycle which may contain one or more double bonds and which is attached via a ring carbon atom or a ring nitrogen atom. Heterocyclyl for its part may also be substituted via C or N. Examples which may be mentioned are: tetrahydrofuryl, pyrrolidinyl, pyrrolinyl, piperidinyl, dihydropyridinyl, piperazinyl, morpholinyl, azepinyl, diazepinyl. Preference is given to piperidinyl, morpholinyl and pyrrolidinyl.

In the context of the invention, preferred salts are physiologically acceptable salts of the compounds according to the invention.

Physiologically acceptable salts of the compounds according to the invention can be acid addition salts of the substances according to the invention with mineral acids, carboxylic acids or sulfonic acids. Particular preference is, for example, given to salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

However, salts that may also be mentioned include salts with customary bases such as, for example, alkali metal salts (for example sodium or potassium salts), alkaline earth metal salts (for example calcium or magnesium salts) or ammonium salts derived from ammonia or organic amines, such as, for example, diethylamine, triethylamine, ethyldiisopropylamine, procaine, dibenzylamine, N-methylmorpholine, dihydroabietylamine, 1-ephenamine or methylpiperidine, or derived from natural amino acids, such as, for example, glycine, lysine, arginine or histidine.

The compounds according to the invention can exist in stereoisomeric forms which are either like image and mirror image (enantiomers) or which are not like image and mirror image (diastereomers). The invention relates both to the enantiomers or diastereomers or to their respective mixtures. The racemates, like the diastereomers, can be separated in a known manner into the stereoisomerically uniform components.

The compounds according to the invention can also be present as prodrugs. This applies in particular to the hydroxyl group on R⁵ which can be esterified without any substantial loss in activity. These include, by way of example and by way of preference, aliphatic esters, for example butyl esters, aromatic esters, for example benzyl esters, or α-amino acid esters, for example succinic acid monoamide.

Preference is given to compounds of the general formula (I) in which
A is attached via position 2, 3, 5 or 6 to the aromatic ring, and
A represents oxygen or NR⁶,
E represents oxygen, CR⁹R¹⁰ or NR⁷,
Y represents oxygen or NR⁸,
D and X are identical or different and represent in each case oxygen or sulfur,
G represents hydrogen,
or
G represents C₆-C₁₀-aryl, where C₆-C₁₀-aryl may optionally be substituted by up to three substituents independently of one another selected from the group consisting of halogen, hydroxyl, nitro, cyano, C₁-C₆-alkoxy, hydroxycarbonyl, C₁-C₆-alkoxycarbonyl, amino, mono- or di-C₁-C₆-alkylamino, mono- or di-C₁-C₆-alkylaminocarbonyl and C₁-C₆-alkyl,
   where
   C₁-C₆-alkoxy, C₁-C₆-alkoxycarbonyl, mono- or di-C₁-C₆-alkylamino, mono- or di-C₁-C₆-alkylaminocarbonyl or C₁-C₆-alkyl may optionally be substituted by up to three substituents independently of one another selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₆-C₁₀-aryl,
or
G represents C₆-C₁₀-aryl, where C₆-C₁₀-aryl may optionally be substituted by phenyl,
   where
   phenyl may optionally be substituted by up to three substituents independently of one another selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₁-C₆-alkyl,
   where
   C₁-C₆-alkyl for its part may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by phenyl,
   where
   phenyl may optionally be substituted by C₅-C₆-heteroaryl or C₅-C₇-heterocyclyl,
   where
   C₅-C₆-heteroaryl or C₅-C₇-heterocyclyl for their part may optionally be substituted by up to three substituents independently of one another selected from the group consisting of halogen, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by a group of the following formula 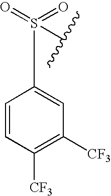
or
G represents C₅-C₁₀-heteroaryl or C₅-C₇-heterocyclyl, where C₅-C₁₀-heteroaryl or C₅-C₇-heterocyclyl may optionally be substituted by up to three substituents independently of one another selected from the group consisting of halogen, nitro, cyano, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₃-C₁₀-cycloalkyl, where C₃-C₁₀-cycloalkyl may optionally be substituted by up to three substituents independently of one another selected from the group consisting of halogen, nitro, cyano, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹, R², R³ and R⁴ are identical or different and represent in each case hydrogen, amino, mono- or di-C₁-C₆-alkylamino, C₁-C₆-alkylcarbonylamino, C₆-C₁₀-aryl or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and
   where C₆-C₁₀-aryl may optionally be substituted by up to three substituents selected from the group consisting of halogen, hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl, mono- or di-C₁-C₆-alkylaminocarbonyl and C₁-C₆-alkyl,
   where
   C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
where R¹, R², R³ and R⁴ are not simultaneously hydrogen,
or
R¹ and R² or R³ and R⁴ together with the carbon atom to which they are attached form a C₃-C₆-cycloalkyl ring, where the C₃-C₆-cycloalkyl ring may optionally be substituted by up to three substituents independently of one another selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R³ together with the carbon atoms to which they are attached form a C₃-C₆-cycloalkyl ring, where the C₃-C₆-cycloalkyl ring may optionally be substituted by up to three substituents independently of one another selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁵ represents hydrogen, halogen, hydroxyl, C₁-C₆-alkoxy, amino, mono- or di-C₁-C₆-alkylamino or C₁-C₆-alkyl, where C₁-C₆-alkoxy, mono- or di-C₁-C₆-alkylamino or C₁-C₆-alkyl may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are identical or different and represent in each case hydrogen or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁹ and R¹⁰ are identical or different and represent in each case hydrogen, NR¹¹R¹², OR¹³ or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹¹, R¹² and R¹³ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
and their tautomers, stereoisomers, stereoisomeric mixtures and their pharmacologically acceptable salts.

Preference is also given to compounds of the general formula (I) in which
A is attached via position 2, 3, 5 or 6 to the aromatic ring and
A represents NR⁶,
E represents NR⁷,
Y represents NR⁸,
D and X represent oxygen,
G represents C₆-C₁₀-aryl, where C₆-C₁₀-aryl may optionally be substituted by up to three substituents independently of one another selected from the group consisting of halogen, hydroxyl, cyano and C₁-C₆-alkyl,
   where
   C₁-C₆-alkyl may optionally be substituted by up to three substituents of halogen,
or
G represents C₅-C₆-heteroaryl, where C₅-C₆-heteroaryl may optionally be substituted by up to three substituents independently of one another selected from the group consisting of halogen and C₁-C₃-alkyl,
or
G represents C₃-C₁₀-cycloalkyl, where C₃-C₁₀-cycloalkyl may optionally be substituted by up to three substituents C₁-C₆-alkyl,
R¹, R² and R³ are identical or different and represent in each case hydrogen or represent C₁-C₃-alkyl,
R⁴ represents hydrogen, C₆-C₁₀-aryl or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and
   where C₆-C₁₀-aryl may optionally be substituted by up to three substituents independently of one another selected from the group consisting of halogen, hydroxyl, C₁-C₆-alkoxy and C₁-C₆-alkyl,
where R¹, R², R³ and R⁴ do not simultaneously represent hydrogen,
R⁵ represents hydrogen, halogen, hydroxyl, amino, mono- or di-C₁-C₆-alkylamino or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ represent hydrogen,
and their tautomers, stereoisomers, stereoisomeric mixtures and their pharmacologically acceptable salts.

Preference is given to compounds of the general formula (I) 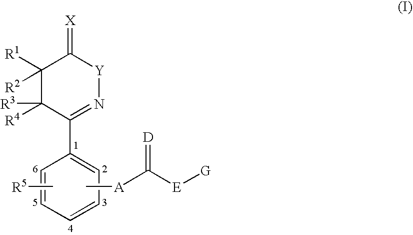
in which
A is attached via position 2, 3, 5 or 6 to the aromatic ring and
A represents NR⁶,
E represents NR⁷,
Y represents NR⁸,
D and X represent oxygen,
G represents C₆-C₁₀-aryl, where C₆-C₁₀-aryl may optionally be substituted by up to three substituents selected from the group consisting of halogen or C₁-C₆-alkyl,
   where
   C₁-C₆-alkyl may optionally be substituted by up to three substituents of halogen, preferably fluorine,
R¹, R², R³ and R⁴ are identical or different and each represent hydrogen or represent C₁-C₃-alkyl,
R⁵ represents hydrogen,
R⁶, R⁷ and R⁸ represent hydrogen,
and their tautomers, stereoisomers, stereoisomeric mixtures and their pharmacologically acceptable salts.

Preference is also given to compounds of the general formula (I) in which
A is attached via position 2, 3, 5 or 6 to the aromatic ring and
A represents NR⁶,
E represents NR⁷,
Y represents NR⁸,
D and X represent oxygen,
G represents C₆-C₁₀-aryl, where C₆-C₁₀-aryl may optionally be substituted by up to three substituents selected from the group consisting of halogen or C₁-C₆-alkyl,
   where
   C₁-C₆-alkyl may optionally be substituted by up to three substituents of halogen, preferably fluorine,
R¹, R², R³ and R⁴ are identical or different and each represent hydrogen or represent C₁-C₃-alkyl,
where R¹, R², R³ and R⁴ are not simultaneously hydrogen,
R⁵ represents hydrogen,
R⁶, R⁷ and R⁸ represent hydrogen,
and their tautomers, stereoisomers, stereoisomeric mixtures and their pharmacologically acceptable salts.

In a further preferred embodiment, in the compounds of the general formula (I) the radical A is attached via position 3 to the aromatic rings.

In a further preferred embodiment, D and X in the compounds of the general formula (I) are oxygen.

In a further preferred embodiment, A, E and Y in the compounds of the general formula (I) are NH.

In a further preferred embodiment, G in the compounds of the general formula (I) is substituted phenyl.

In a further preferred embodiment, R¹, R² and R⁵ in the compounds of the general formula (I) are hydrogen, and R³ and R⁴ are methyl.

In a further preferred embodiment, R⁵ in the compounds of the general formula (I) is hydrogen, hydroxyl, chlorine or fluorine.

The invention furthermore relates to processes for preparing the compounds of the formula (I).

In process
[A] compounds of the general formula (II) 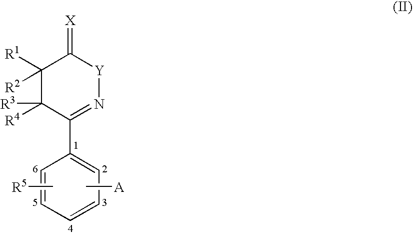
   in which
A is attached via position 2, 3, 5 or 6 to the aromatic ring and
R¹, R², R³, R⁴, R⁵, A, X and Y are as defined above
are reacted with compounds of the general formula (III)
   D=C═N-G (III)
in which
D and G are as defined above
to give compounds of the general formula (Ia) 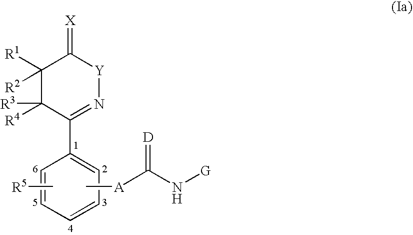
in which
A is attached via position 2, 3, 5 or 6 to the aromatic ring and
R¹, R², R³, R⁴, R⁵, A, D, G, X and Y are as defined above,
in inert solvents which include halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, hyrocarbons, such as benzene, xylene, toluene, hexane, cyclohexane or mineral oil fractions, or other solvents, such as ethyl acetate, acetone, dimethylformamide, dimethylacetamide, 2-butanone, dimethyl sulfoxide, acetonitrile or pyridine, preferred solvents being tetrahydrofuran or methylene chloride, if appropriate in the presence of a base, such as, for example, alkali metal carbonates, such as cesium carbonate, sodium carbonate or potassium carbonate, or potassium tert-butoxide, or other bases, such as sodium hydride, DBU, triethylamine or diisopropylethylamine, preferably triethylamine, preferably in a temperature range of from room temperature to the reflux temperature of the solvents, at atmospheric pressure.

Below, the compounds of the general formula (II) are represented as (IIa), (IIb) and (IIc).

The compounds of the general formula (III) are known or can be synthesized by known processes from the corresponding starting materials.

In Process
[B] compounds of the general formula (II) are reacted with compounds of the general formula (IV) 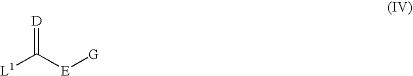
   in which
D, E and G are as defined above and
L¹ represents p-nitrophenyl or halogen, preferably bromine or chlorine,
to give compounds of the general formula (I) 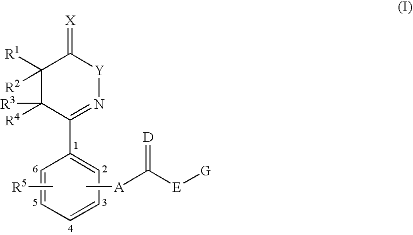
in which
A is attached via position 2, 3, 5 or 6 to the aromatic ring and
R¹, R², R³, R⁴, R⁵, A, D, E, G, X and Y are as defined above,
in inert solvents which include halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, hyrocarbons, such as benzene, xylene, toluene, hexane, cyclohexane or mineral oil fractions, or other solvents, such as ethyl acetate, acetone, dimethylformamide, dimethylacetamide, 2-butanone, acetonitrile or pyridine, preferred solvents being tetrahydrofuran or methylene chloride, if appropriate in the presence of a base, such as, for example, alkali metal carbonates, such as cesium carbonate, sodium carbonate or potassium carbonate, or potassium tert-butoxide, or other bases, such as sodium hydride, DBU, triethylamine or diisopropylethylamine, preferably triethylamine, preferably in a temperature range of from room temperature to the reflux temperature of the solvents, at atmospheric pressure.

The compounds of the general formula (IV) are known or can be prepared by known processes from the corresponding starting materials

In process
[C] compounds of the general formula (V) 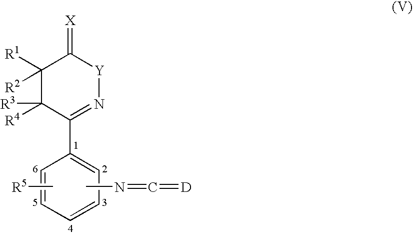
   in which
—NCD is attached via position 2, 3, 5 or 6 to the aromatic ring and
R¹, R², R³, R⁴, R⁵, D, X and Y are as defined above
are reacted with compounds of the general formula (VI)
   H-M-G (VI)
in which
G is as defined above and
M represents oxygen or NR⁷,
   where
   R⁷ is as defined above,
to give compounds of the general formula (Ib) 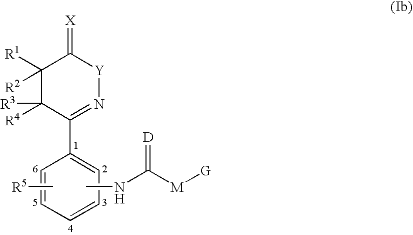
in which
—NH—C(D)-M-G is attached via position 2, 3, 5 or 6 to the aromatic ring and
R¹, R², R³, R⁴, R⁵, D, G, M, X and Y are as defined above,
in inert solvents which include halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, hyrocarbons, such as benzene, xylene, toluene, hexane, cyclohexane or mineral oil fractions, or other solvents, such as ethyl acetate, acetone, dimethylformamide, dimethylacetamide, 2-butanone, dimethyl sulfoxide, acetonitrile or pyridine, preferred solvents being tetrahydrofuran or methylene chloride, if appropriate in the presence of a base, such as, for example, alkali metal carbonates, such as cesium carbonate, sodium carbonate or potassium carbonate, or potassium tert-butoxide, or other bases, such as sodium hydride, DBU, triethylamine or diisopropylethylamine, preferably triethylamine, preferably in a temperature range of from room temperature to the reflux temperature of the solvents, at atmospheric pressure.

The compounds of the general formula (VI) are known or can be synthesized by known processes from the corresponding starting materials.

To prepare the compounds of the general formula (IIa) 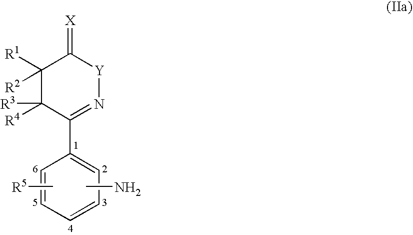
in which
NH₂ is attached via position 2, 3, 5 or 6 to the aromatic ring and
R¹, R², R³, R⁴, R⁵, X and Y are as defined above,
compounds of the general formula (VII) 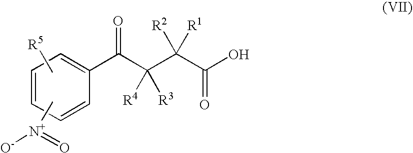
in which
NO₂ is attached via position 2, 3, 5 or 6 to the aromatic ring and
R¹, R², R³, R⁴ and R⁵ are as defined above
are, if X represents oxygen,
initially reacted with hydrazine, hydroxylamine or a compound of the general formula (VIII) 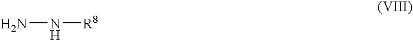
in which
R⁸ is as defined above,
and the nitro group is then reduced to the amino group. These two reactions can take place in one or two reaction steps.

In a one-step process, the reaction is carried out simultaneously with hydrazine and with palladium on carbon in inert solvents including ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, n-propanol, isopropanol, n-butanol or tert-butanol, hydrocarbons, such as benzene, xylene, toluene, hexane, cyclohexane or mineral oil fractions, or other solvents, such as dimethylacetamide, acetonitrile or pyridine, preferred solvents being ethanol or isopropanol, preferably in a temperature range of from room temperature to the reflux temperature of the solvents, at atmospheric pressure.

In a two-step process, the reaction is initially carried out using hydrazine, hydroxylamine or a compound of the general formula (VIII) in inert solvents including ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, dimethylacetamide, acetonitrile or pyridine, preferred solvents being ethanol or isopropanol, preferably in a temperature range of from room temperature to the reflux temperature of the solvents, at atmospheric pressure.

In the second step, the reaction is carried out with hydrogen donors, preferably hydrazine or hydrogen and with palladium on carbon, or with tin chloride in inert solvents including ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as ethyl acetate, dimethylformamide, dimethylacetamide, acetonitrile or pyridine, preferred solvents being ethanol, isopropanol or, in the case of tin dichloride, in dimethylformamide, preferably in a temperature range of from room temperature to the reflux temperature of the solvents, at from atmospheric pressure to 3 bar.

If X is sulfur
the compound is initially reacted with hydrazine, hydroxylamine or a compound of the general formula (VIII), the oxygen is then changed for sulfur using Lawesson's reagent and the nitro group is subsequently reduced to an amino group.

In the first step, the reaction is carried out with hydrazine, hydroxylamine or a compound of the general formula (VIII) in inert solvents including ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, dimethylacetamide, acetonitrile or pyridine, preferred solvents being ethanol or isopropanol, preferably in a temperature range of from room temperature to the reflux temperature of the solvents, at atmospheric pressure.

In the second step, the reaction is carried out with Lawesson's reagent in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as nitromethane, 1,2-dimethoxyethane, dimethyl sulfoxide or pyridine, preferably toluene, xylene or dioxane, preferably in a temperature range of from room temperature to the reflux temperature of the solvents, at atmospheric pressure.

In the third step, the reaction is carried out with hydrogen donors, preferably hydrazine or hydrogen and with palladium on carbon, or with tin dichloride in inert solvents including ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, dimethylacetamide, acetonitrile, pyridine, preferred solvents being ethanol, isopropanol or, in the case of tin dichloride, in dimethylformamide, preferably in a temperature range of from room temperature to the reflux temperature of the solvents, at from atmospheric pressure to 3 bar.

Compounds of the general formula (VII) can be present in two different forms. In the description of the processes, only the open-chain form is shown.

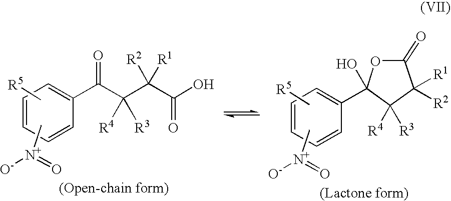

To prepare the compounds of the general formula (IIb) 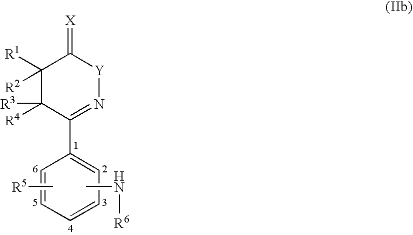
in which
NHR⁶ is attached via position 2, 3, 5 or 6 to the aromatic ring and
R¹, R², R³, R⁴, R⁵, R⁶, X and Y are as defined above,
compounds of the general formula (IIa) are reacted with compounds of the general formula (IX)
   L²-R⁶ (IX)
in which
R⁶ is as defined above and
L² represents halogen, preferably bromine or iodine,
in inert solvents including ethers, such as diethyl ether, methyl tert-butyl ether, 1,2-dimethoxyethane, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethylacetamide, acetonitrile or pyridine, preferred solvents being tetrahydrofuran or diethyl ether, if appropriate in the presence of a base, such as, for example, alkali metal hydroxides, such as sodium hydroxide or potassium hydroxide, or alkali metal carbonates, such as cesium carbonate, sodium carbonate or potassium carbonate, or amides, such as sodium amide, lithium bis(trimethylsilyl)amide, lithium diisopropylamide, or organometallic compounds, such as butyllithium or phenyllithium, or other bases, such as sodium hydride, DBU, triethylamine or diisopropylethylamine, preferably diisopropylethylamine, potassium tert-butoxide or DBU, preferably in a temperature range of from room temperature to the reflux temperature of the solvents, at atmospheric pressure.

The compounds of the general formula (IX) are known or can be synthesized from the corresponding starting materials by known processes.

To prepare compounds of the general formula (IIc) 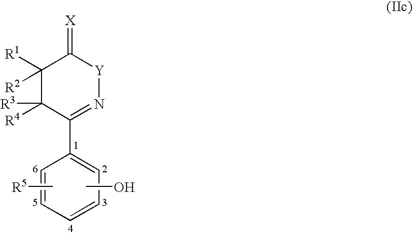
in which
OH is attached via position 2, 3, 5 or 6 to the aromatic ring and
R¹, R², R³, R⁴, R⁵, X and Y are as defined above,
initially the diazonium compounds are prepared from compounds of the general formula (IIa) according to methods known to the person skilled in the art, and these diazonium compounds are then heated to give the phenols (cf. Organikum, 17th edition, VEB Deutscher Verlag der Wissenschaften, Berlin, p. 543).

To prepare compounds of the general formula (V), compounds of the general formula (IIa)
are reacted with trichloromethyl chloroformate
in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as ethyl acetate, acetone, dimethylformamide, dimethylacetamide, 2-butanone, acetonitrile or pyridine. Preferred solvents are tetrahydrofuran or dichloromethane, if appropriate in the presence of a base, such as, for example, 1,8-bis(dimethylamino)naphthalene, DBU, triethylamine or diisopropylethylamine, preferably 1,8-bis(dimethylamino)-naphthalene, preferably in a temperature range of from room temperature to the reflux temperature of the solvents, at atmospheric pressure.

To prepare compounds of the general formula (VII), compounds of the general formula (X) 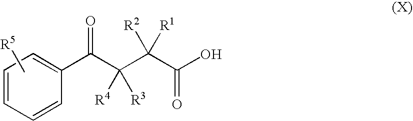
in which
R¹, R², R³, R⁴ and R⁵ are as defined above
are reacted with fuming nitric acid, concentrated nitric acid or nitrating acid, preferably in a temperature range of from −30° C. to 0° C. at atmospheric pressure.

Compounds of the general formula (X) can be present in two different forms. In the description of the processes, only the open-chain form is shown.

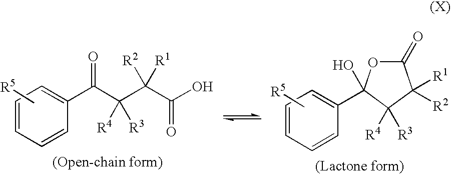

To prepare the compounds of the general formula (X), compounds of the general formula (XI) 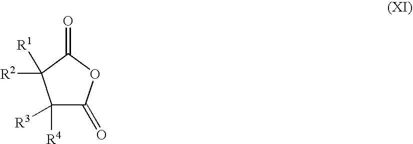
in which
R¹, R², R³ and R⁴ are as defined above
are reacted with compounds of the general formula (XII) 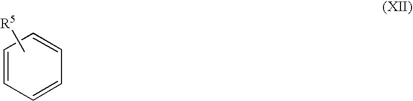
in which
R⁵ is as defined above
with Lewis acids, preferably aluminum trichloride,
in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, hydrocarbons, such as benzene, nitrobenzene, hexane, cyclohexane or mineral oil fractions, or other solvents, such as ethyl acetate, acetone, dimethylformamide, dimethylacetamide, 2-butanone, dimethyl sulfoxide, acetonitrile or pyridine (the preferred solvent being 1,2-dichloroethane), preferably in a temperature range of from −20° C. to room temperature at atmospheric pressure.

The compounds of the general formulae (XI) and (XII) are known or can be synthesized from the corresponding starting materials by known processes.

In an alternative synthesis route, to prepare the compounds of the general formula (Xa), these compounds being compounds of the general formula (X), in which
R² represents hydrogen,
compounds of the general formula (XIIIa) 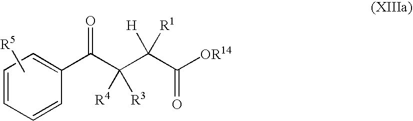
in which
R¹, R³, R⁴ and R⁵ are as defined above
R¹⁴ represents (C₁-C₆)-alkyl, preferably methyl or ethyl,
are reacted with bases, such as, for example, alkali metal hydroxides, such as sodium hydroxide, lithium hydroxide or potassium hydroxide, or alkali metal carbonates, such as cesium carbonate, sodium carbonate or potassium carbonate, preferably sodium hydroxide, in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, dimethylacetamide, dimethyl sulfoxide, acetonitrile or pyridine, or mixture of solvents (preferred solvents being tetrahydrofuran and/or methanol), preferably in a temperature range of from 0° C. to room temperature at atmospheric pressure.

The compounds of the general formula (X) can also be prepared analogously to the synthesis route described for processes of compounds of the general formula (Xa) from compounds of the general formula (XIII).

To prepare the compounds of the general formula (XIIIa), compounds of the general formula (XIV) 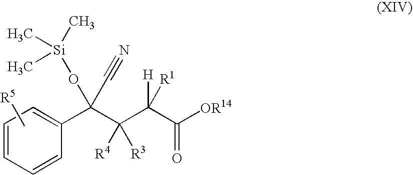
in which
R¹, R³, R⁴, R⁵ and R¹⁴ are as defined above
are reacted with tetrabutylammonium fluoride
in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, ethyl acetate, acetone, dimethylformamide, dimethylacetamide, 2-butanone, dimethyl sulfoxide, acetonitrile or pyridine (the preferred solvent being tetrahydrofuran), preferably in a temperature range of from 0° C. to room temperature at atmospheric pressure.

To prepare the compounds of the general formula (XIV), compounds of the general formula (XV) 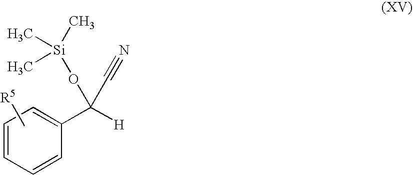
in which
R⁵ is as defined above
are reacted with compounds of the general formula (XVI) 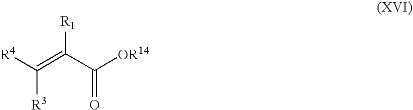
in which
R¹, R³, R⁴ and R¹⁴ are as defined above
in inert solvents including ethers, such as diethyl ether, methyl tert-butyl ether, 1,2-dimethoxyethane, dioxane, tetrahydrofuran, glycol dimethyl ether or diethylene glycol dimethyl ether, hydrocarbons, such as benzene, ethylbenzene, xylene, toluene, hexane, heptane, cyclohexane or mineral oil fractions, or other solvents, such as dimethylformamide, dimethylacetamide, acetonitrile or pyridine, or mixtures of the solvents, preferred solvents being diethyl ether, tetrahydrofuran, heptane and/or ethylbenzene, if appropriate in the presence of a base, such as, for example, alkali metal hydroxides, such as sodium hydroxide or potassium hydroxide, or alkali metal carbonates, such as cesium carbonate, sodium carbonate or potassium carbonate, or sodium methoxide or potassium methoxide, or sodium ethoxide or potassium ethoxide, or potassium tert-butoxide, or amides, such as sodium amide, lithium bis(trimethylsilyl)amide, lithium diisopropylamide, or organometallic compounds, such as butyllithium or phenyllithium, or other bases, such as sodium hydride, DBU, triethylamine or diisopropylethylamine, preferably lithium diisopropylamide, preferably in a temperature range of from −78° C. to room temperature at atmospheric pressure.

The compounds of the general formula (XVI) are known or can be prepared from the corresponding starting materials by known methods.

To prepare the compounds of the general formula (XV), compounds of the general formula (XVII) 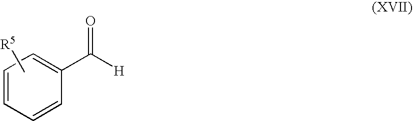
in which
R⁵ is as defined above
are reacted with trimethylsilyl cyanide and zinc iodide
if appropriate in inert solvents including halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, ethyl acetate, acetone, dimethylformamide, dimethylacetamide, 2-butanone, dimethyl sulfoxide, acetonitrile or pyridine (the preferred solvent being tetrahydrofuran), preferably in a temperature range of from room temperature to 100° C. at atmospheric pressure.

The compounds of the general formula (XVII) are known or can be synthesized from the corresponding starting materials by known processes.

To prepare the compounds of the general formula (XIII) 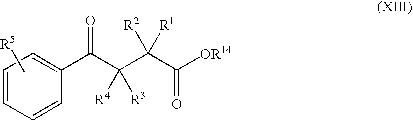
in which
R¹, R², R³, R⁴, R⁵ and R¹⁴ are as defined above,
compounds of the general formula (XVIII) 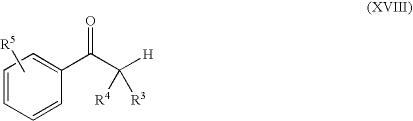
in which
R³, R⁴ and R⁵ are as defined above,
are reacted with compounds of the general formula (XIX) 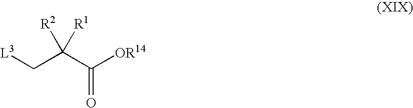
in which
R¹, R² and R¹⁴ are as defined above and
L³ represents halogen, preferably bromine or iodine,
in inert solvents including ethers, such as diethyl ether, methyl tert-butyl ether, 1,2-dimethoxyethane, dioxane, tetrahydrofuran, glycol dimethyl ether or diethylene glycol dimethyl ether, hydrocarbons, such as benzene, ethylbenzene, xylene, toluene, preferred solvents being tetrahydrofuran or toluene, if appropriate in the presence of a base, such as, for example, amides, such as sodium amide, lithium hexamethyldisilazide, potassium hexamethyldisilazide, lithium diisopropylamide or other bases, such as sodium hydride, DBU or diisopropylethylamine, preferably sodium amide, lithium hexamethyldisilazide, potassium hexamethyldisilazide or lithium diisopropylamide, preferably in a temperature range of from −78° C. to room temperature at atmospheric pressure.

The compounds of the general formulae (XVIII) and (XIX) are known or can be synthesized from the corresponding starting materials by known processes (for (XVIII) cf. M. R. Schneider, H. Ball, J. Med. Chem. 1986, 29, 75-79; Robl, et al., Synthesis 1991, 56; J. Org. Chem. 1996, 61, 607).

In an alternative synthesis route for preparing compounds of the general formula (IIaa), these compounds being compounds of the general formula (IIa) in which
R¹ and R² represent hydrogen,
compounds of the general formula (XX) 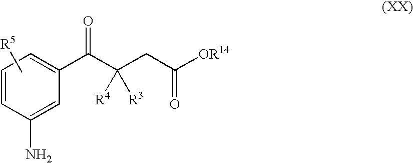
in which
R³, R⁴, R⁵ and R¹⁴ are as defined above
are reacted with hydrazine. The reaction is carried out analogously to the first step of the two-step process described for preparing the compounds of the general formula (IIa).
To prepare the compounds of the general formula (XX), compounds of the general formula (XXI) 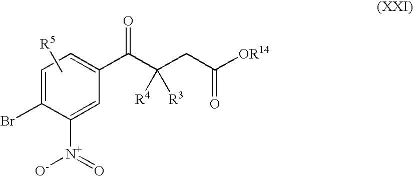
in which
R³, R⁴, R⁵ and R¹⁴ are as defined above
are reacted with reducing agents. The reaction is carried out analogously to the second step of the two-step process described for preparing the compounds of the general formula (IIa).

To prepare the compounds of the general formula (XXI), compounds of the general formula (XXII) 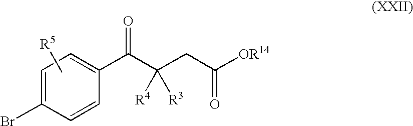
in which
R³, R⁴, R⁵ and R¹⁴ are as defined above
are reacted with fuming nitric acid, concentrated nitric acid or nitrating acid analogously to the process described for preparing the compounds of the general formula (VII).

The compounds of the general formula (XXII) can be synthesized from the corresponding starting materials by the process described for the compounds of the general formula (XIII).

In an alternative synthesis route for preparing the compounds of the general formula (XX), compounds of the general formula (XXIII) 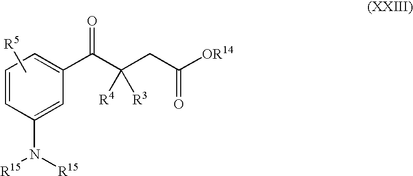
in which
R³, R⁴, R⁵ and R¹⁴ are as defined above
and
R¹⁵ represents allyl or benzyl
are, in the case of benzyl, reacted with reducing agents. The reaction is carried out analogously to the second step of the two-step process described for preparing the compounds of the general formula (IIa).

In the case of allyl, a process with tetrakistriphenylphosphinepalladium and N,N-dimethylbarbituric acid is used, cf. F. Garro-Helion, A. Merzouk, F. Guibe, J. Org. Chem. 1993, 58, 6109-6113.

The compounds of the general formula (XXIII) can be prepared from the corresponding starting materials by the process described for the compounds of the general formula (XIII).

The processes described above can be illustrated in an exemplary manner by the formula schemes below:

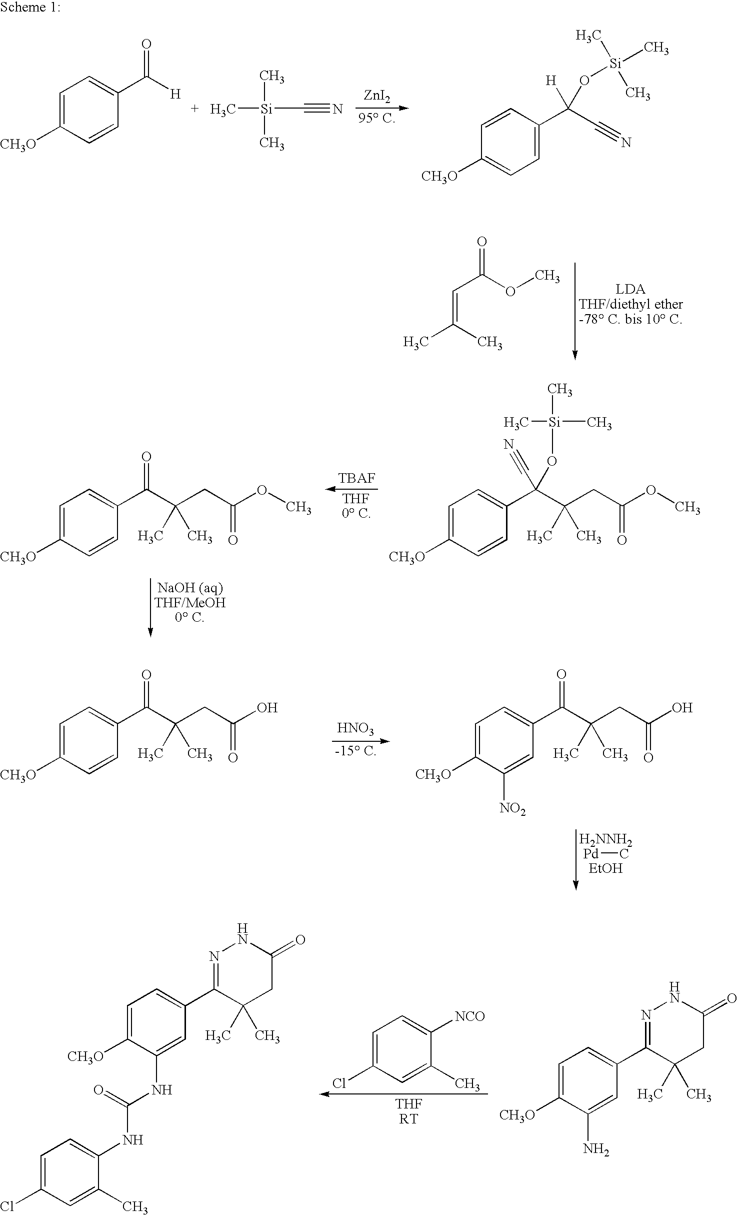

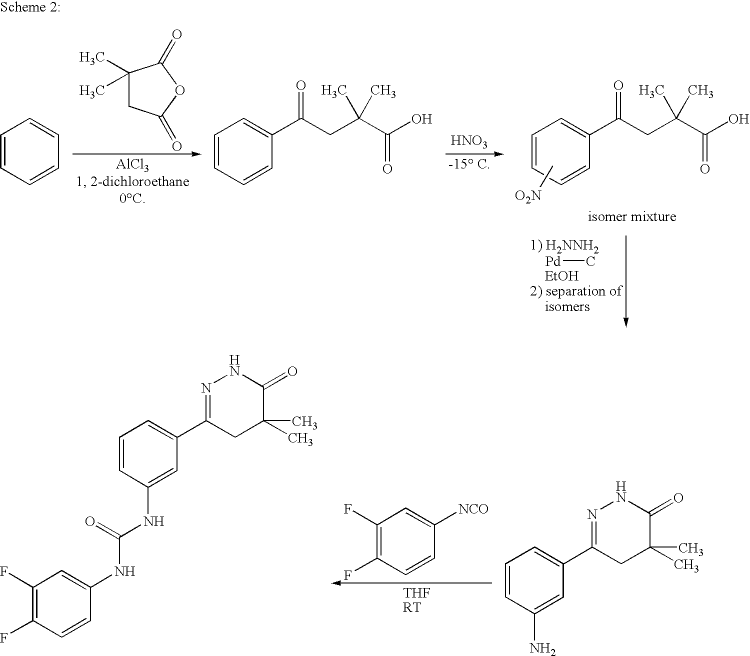

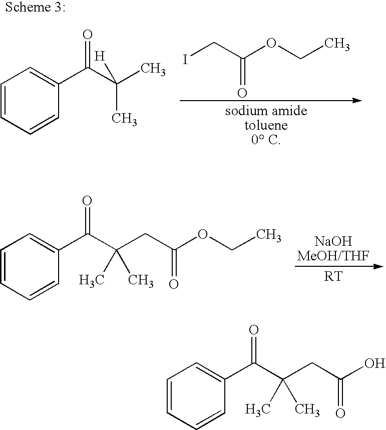

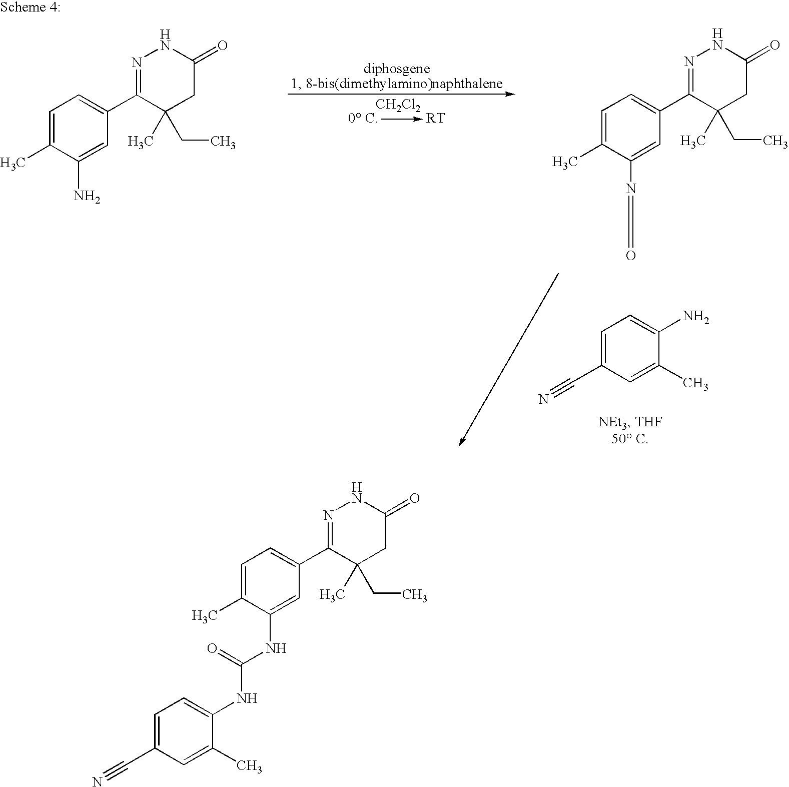

The compounds of the general formula (I) according to the invention have a surprising activity spectrum which could not have been foreseen. They show antiviral action against representatives of the group of the Herpes viridae, in particular against the human cytomegalovirus (HCMV). Accordingly, they are suitable for the treatment and prophylaxis of disorders caused by Herpes viridae, in particular of disorders caused by human cytomegaloviruses.

Owing to their particular properties, the compounds of the general formula (I) can be used for preparing medicaments suitable for the prophylaxis or treatment of diseases, in particular viral disorders.

Owing to their properties, the compounds according to the invention are useful active compounds for the treatment and prophylaxis of human cytomegalovirus infections and disorders caused by these. Areas of indication which may be mentioned are, for example:
1) The treatment and prophylaxis of HCMV infections in AIDS patients (retinitis, pneumonitis, gastrointestinal infections).
2) The treatment and prophylaxis of cytomegalovirus infections in bone marrow and organ transplant patients suffering from HCMV pneumonitis, HCMV encephalitis and gastrointestinal and systemic HCMV infections, which are frequently life-threatening.
3) The treatment and prophylaxis of HCMV infections in neonates and infants.
4) The treatment of an acute HCMV infection in pregnant women.
5) The treatment of an HCMV infection in immunosuppressed patients suffering from cancer and undergoing cancer therapy.

The novel active compounds can be used on their own and, if required, also in combination with other antivirally active compounds, such as, for example, gancyclovir or acyclovir.

Descriptions of Biological Tests:

In vitro Action:

Anti-HCMV (Anti-Human Cytomegalovirus) Cytopathogenicity Tests:

The test compounds are used as 50 millimolar (mM) solutions in dimethyl sulfoxide (DMSO). The reference compounds used are ganciclovir, foscarnet and cidofovir. In each case 2 μl of the 50, 5, 0.5 and 0.05 mM DMSO stock solutions are added to in each case 98 μl of cell culture medium in row 2 A-H in duplicate, and 1:2 dilutions with in each case 50 μl of medium are then made up to row 11 of the 96-well plate.

The wells in rows 1 and 12 each contain 50 μl of medium. In each case 150 μl of a suspension of 1×10⁴ cells (NHDF=normal human dermal fibroblasts) (row 1 =cell control) and, for rows 2-12, a mixture of HCMV-infected and non-infected NHDF cells (M.O.I.=0.001-0.002), i.e. 1-2 infected cells per 1000 non-infected cells, are then pipetted into the wells. Row 12 (without substance) serves as virus control. The final test concentrations are 250-0.0005 μM. The plates are incubated at 37° C./5% CO₂ for 6 days, i.e. until all cells in the virus controls are infected (100% cytopathogenic effect [CPE]). The wells are then fixed by adding a mixture of formalin and Giemsa's dye and stained (30 minutes), washed with doubly distilled water and dried in a drying cabinet at 50° C. The plates are then examined visually using an overhead microscope (Plaque multiplier from Technomara).

Using the test plates, the following data can be determined:
CC₅₀ (NHDF)=substance concentration in μM where, compared to the untreated cell control, no cytostatic effects on the cells are evident;
EC₅₀ (HCMV)=substance concentration in μM where the CPE (cytopathic effect) is inhibited by 50% compared to the untreated virus control;
SI (selectivity index)=CC₅₀ (NHDF)/EC₅₀ (HCMV).
   In vivo Action:
   HCMV Xenograft Gelfoam® Model:
   Animals:

Female immunodeficient mice (16-18 g), Fox Chase SCID or Fox Chase SCID-NOD or SCID-beige, 3-4 weeks old, are obtained from commercial breeders (Bomholtgaard, Jackson). The animals are kept under sterile conditions (including litter and feed) in isolation units.

Virus Culture:

Human cytomegalovirus (HCMV), DavisSmith strain, is cultivated in vitro on embryonal human dermal fibroblasts (NHDF cells). Following infection of the NHDF cells with a multiplicity of infection (M.O.I.) of 0.01, the virus-infected cells are harvested after 5-7 days and, in the presence of minimal essential medium (MEM), 10% fetal calf serum (FCS) with 10% DMSO, stored at −40° C. Following serial dilution of the virus-infected cells in steps of ten, the titer is determined on 24-well plates of confluent NHDF cells after vital stain with Neutral Red.

Preparation of the Sponges, Transplantation, Treatment and Evaluation:

Collagen sponges (Gelfoam®; from Peasel & Lorey, order No. 40734; K. T. Chong et al., Abstracts of 39th Interscience Conference on Antimicrobial Agents and Chemotherapy, 1999, p. 439) of a size of 1×1×1 cm are initially wetted with phosphate-buffered saline (PBS), the enclosed air bubbles are removed by degassing and the sponges are then stored in MEM+10% FCS. 3 hours after infection, 1×10⁶ virus-infected NHDF cells (infection with HCMV-Davis M.O.I.=0.01) are detached and, in 20 μl of MEM and 10% FCS, added dropwise to a moist sponge. 12-13 hours later, the infected sponges are incubated with 25 μl PBS/0.1% BSA/1 mM DTT with 5 ng/μl of basic fibroblast growth factor (bFGF). For transplantation, the immunodeficient mice are anesthetized with Avertin, the dorsal hair is removed using a dry shaver, the epidermis is opened 1-2 cm and relaxed and the moist sponges are transplanted under the dorsal skin. The operation wound is closed using tissue adhesive. 24 hours after the transplantation, the mice are treated perorally with substance three times a day (7 a.m., 2 p.m. and 7 p.m.) for a period of 8 days. The dose is 7 or 15 or 30 or 60 mg/kg of body weight, the application volume is 10 ml/kg of body weight. The substances are formulated in the form of a 0.5% strength-suspension in tylose with 2% DMSO. 9 days after the transplantation and 16 hours after the last administration of substance, the animals are killed painlessly and the sponge is removed. The virus-infected cells are released from the sponge by digestion with collagenase (330 U/1.5 ml) and stored in the presence of MEM, 10% fetal calf serum, 10% DMSO at −140° C. Evaluation is carried out after serial dilution of the virus-infected cells in steps of 10 by titer determination on 24-well plates of confluent NHDF cells after vital stain with Neutral Red. What is determined is the number of infectious virus particles after substance treatment compared to the placebo-treated control group.

Representative activity data for the compounds according to the invention are given in Table 1:

TABLE 1

| | NHDF | HCMV | | Murine xenograft model |
|---|---|---|---|---|
| Example | CC50 | EC50 | SI | ED50 [mg]kg] |
| No. | [μM] | [μM] | HCMV | t.i.d. |
| 1 | 125 | 0.96 | 130 | 18 |
| 2 | 23 | 0.07 | 329 | n.d. |
| 3 | 24 | 0.13 | 185 | n.d. |
| 4 | >23 | 0.15 | >153 | 8 |
| 5 | 125 | 6.10 | 21 | n.d. |

The novel active compounds can be converted in a known manner into the customary formulations, such as tablets, coated tablets, pills, granules, aerosols, syrups, emulsions, suspensions and solutions, using inert non-toxic, pharmaceutically acceptable carriers or solvents. Here, the therapeutically active compound should in each case be present in a concentration of about 0.5 to 90% by weight of the total mixture, i.e. in amounts which are sufficient in order to achieve the dosage range indicated.

The formulations are prepared, for example, by extending the active compounds using solvents and/or carriers, if appropriate using emulsifiers and/or dispersants, it optionally being possible, for example, to use organic solvents as auxiliary solvents if the diluent used is water.

Administration is carried out in a customary manner, preferably orally, parenterally or topically, in particular perlingually or intravenously.

In the case of parenteral administration, solutions of the active compounds can be employed using suitable liquid carrier materials.

In general, it has proved advantageous in the case of intravenous administration to administer amounts of about 0.001 to 10 mg/kg, preferably about 0.01 to 5 mg/kg, of body weight, to achieve effective results, and in the case of oral administration, the dosage is about 0.01 to 25 mg/kg, preferably 0.1 to 10 mg/kg of body weight.

In spite of this, it may be necessary to depart from the amounts mentioned, namely depending on the body weight or on the type of administration route, on the individual response toward the medicament, the type of its formulation and the time or interval at which administration takes place. Thus, in some cases it may be adequate to manage with less than the abovementioned minimum amount, while in other cases the upper limit mentioned has to be exceeded. In the case of the administration of relatively large amounts, it may be advisable to divide these into several individual doses over the course of the day.

Abbreviations:

| | |
|---|---|
| abs. | absolute |
| Ac | acetyl |
| acac | acetylacetonyl |
| AIBN | α,α′-azobis(isobutyronitrile) |
| Aloc | allyloxycarbonyl |
| aq. | aqueous |
| 9-BBN | 9-borabicyclo[3.3.1]nonane |
| Bn | benzyl |
| Boc | tert-butoxycarbonyl |
| Bom | benzyloxymethyl |
| BOP | benzotriazol-1-yloxy-tris(dimethylamino)phosphonium |
| | hexafluorophosphate |
| Bu | butyl |
| Bz | benzoyl |
| CAN | cerium ammonium nitrate |
| Cbz | benzyloxycarbonyl |
| CDI | N,N′-carbonyldiimidazole |
| CH | cyclohexane |
| Cp | cyclopentadienyl |
| CSA | 10-camphorsulfonic acid |
| Dabco | 1,4-diazabicyclo[2.2.2]octane |
| DAST | diethylaminosulfur trifluoride |
| DBN | 1,5-diazabicyclo[4.3.0]non-5-ene |
| DBU | 1,8-diazabicyclo[5.4.0]undec-7-ene |
| TLC | thin-layer chromatography |
| DCC | N,N′-dicyclohexylcarbodiimide |
| DCE | 1,2-dichloroethane |
| DCI | direct chemical ionization (MS) |
| DCM | dichloromethane |
| DDQ | 2,3-dichloro-5,6-dicyano-1,4-benzoquinone |
| DEAD | diethyl azodicarboxylate |
| d.e. | diastereomeric excess |
| dist. | distilled |
| DHP | 3,4-dihydro-2H-pyran |
| DIAD | diisopropyl azodicarboxylate |
| DIBAH | diisobutylaluminum hydride |
| DIC | diisopropylcarbodiimide |
| DIEA | N,N-diisopropylethylamine |
| DMA | N,N-dimethylacetamide |
| DMAP | 4-N,N-dimethylaminopyridine |
| DME | 1,2-dimethoxyethane |
| DMF | N,N-dimethylformamide |
| DMPU | N,N′-dimethylpropyleneurea |
| DMSO | dimethyl sulfoxide |
| DNPH | 2,4-dinitrophenylhydrazine |
| DPPA | diphenylphosphoryl azide |
| EDC | N′-(3-dimethylaminopropyl)-N- |
| | ethylcarbodiimide × HCl |
| e.e. | enantiomeric excess |
| EA | ethyl acetate |
| EI | electron impulse ionization (MS) |
| eq | equivalent(s) |
| ESI | electrospray ionization (MS) |
| Et | ethyl |
| liq. | liquid |
| Fmoc | fluorenylmethoxycarbonyl |
| m.p. | melting point |
| fr. | fraction |
| GC | gas chromatography |
| sat. | saturated |
| HATU | O-(7-azabenzotriazol-1-yl)-N,N,N′, |
| | N′-tetramethyluronium hexafluorophosphate |
| HBTU | O-(benzotriazol-1-yl)-N,N,N′,N′-tetramethyluronium |
| | hexafluorophosphate |
| HMDS | 1,1,1,3,3,3-hexamethyldisilazane |
| HMPA or HMPT | hexamethylphosphoric triamide |
| HOBt | 1-hydroxy-1H-benzotriazole × H₂O |
| HOSu | N-hydroxysuccinimide |
| HPLC | high pressure, high performance liquid |
| | chromatography |
| Im | imidazol-1-yl |
| IR | infrared spectroscopy |
| conc. | concentrated |
| b.p. | boiling point |
| cryst. | crystalline/crystallized |
| LAH | lithium aluminum hydride |
| LC-MS | liquid-chromatography-coupled mass spectroscopy |
| LDA | lithium N,N-diisopropylamide |
| LiHMDS | lithium N,N-bistrimethylsilylamide |
| lit. | literature (reference) |
| sol. | solution |
| m | meta |
| mCPBA | meta-chloroperbenzoic acid |
| Me | methyl |
| MEK | methyl ethyl ketone |
| MEM | methoxyethoxymethyl |
| MW | molecular weight |
| MOM | methoxymethyl |
| MPLC | medium-pressure liquid chromatography |
| Ms | methanesulfonyl (mesyl) |
| MS | mass spectroscopy |
| MTBE | methyl tert-butyl ether |
| NBS | N-bromosuccinimade |
| NCS | N-chlorosuccinimide |
| prec. | precipitate |
| NIS | N-iodosuccinimide |
| NMM | N-methylmorpholine |
| NMO | N-methylmorpholine N-oxide |
| NMR | nuclear magnetic resonance spectroscopy |
| o | ortho |
| p | para |
| p.a. | pro analysi |
| PCC | pyridinium chlorochromate |
| PDC | pyridinium dichromate |
| Pfp | pentafluorophenyl |
| Ph | phenyl |
| Piv | pivaloyl |
| PMB | p-methoxybenzyl |
| PNB | p-nitrobenzyl |
| PPA | polyphosphoric acid |
| PPTS | pyridinium p-toluenesulfonate |
| Pr | propyl |
| PS | polystyrene (resin) |
| py | pyridine |
| PyBOP | benzotriazol-1-yloxy-tris(pyrrolidino)phoshonium |
| | hexafluorophosphate |
| RF | reflux |
| R_{f} | retention index (TLC) |
| RP | reverse phase (HPLC) |
| RT | room temperature |
| Rₜ | retention time (HPLC) |
| SEM | 2-(trimethylsilyl)ethoxymethyl |
| subl. | sublimed |
| TBAF | tetrabutylammonium fluoride |
| TBAI | tetrabutylammonium iodide |
| TBDMS | tert-butyldimethylsilyl |
| TBDPS | tert-butyldiphenylsilyl |
| TBTU | O-(benzotriazol-1-yl)-N,N,N′,N′-tetramethyluronium |
| | tetrafluoroborate |
| TEA | triethylamine |
| techn. | technical grade |
| Teoc | 2-(trimethylsilyl)ethoxycarbonyl |
| TES | triethylsilyl |
| Tf | trifluoromethanesulfonyl |
| TFA | trifluoroacetic acid |
| TFAA | trifluoroacetic anhydride |
| TfOH | trifluoromethanesulfonic acid |
| THF | tetrahydrofuran |
| THP | tetrahydropyranyl |
| TIPS | triisopropylsilyl |
| titr. | titrated |
| TMEDA | N,N,N′,N′-tetramethylethylenediamine |
| TMOF | trimethyl orthoformate |
| TMS | trimethylsilyl |
| TPP | triphenylphosphine |
| TPPO | triphenylphosphine oxide |
| Trt | trityl |
| Ts | p-toluenesulfonyl (tosyl) |
| TsOH | p-toluenesulfonic acid |
| v/v | volume-to-volume ratio (of a solution) |
| dil. | diluted |
| cf. | compare |
| vol. | volume |
| w/w | weight-to-weight ratio (of a solution) |
| aq. | aqueous |
| Z | benzyloxycarbonyl |
| decomp. | decomposition |

Starting Materials:
General Procedure [A]:
Synthesis of TMS-cyanohydrins

Under an atmosphere of argon, in a 100 ml three-necked flask dried thoroughly by heating, a spatula tip of anhydrous zinc iodide is added to 55 mmol of trimethylsilyl cyanide. At RT, 50 mmol of the liquid aldehydes are slowly added dropwise (exothermic reaction) (solid aldehydes are added a little at a time as a solid at 60° C.). The resulting brown reaction mixture is heated at 95° C. for 7-8 hours. The product is then distilled under high vacuum in a kugelrohr oven. The resulting colorless or slightly yellow liquids are used without further purification for the subsequent reactions.

The following compounds were prepared according to this procedure:

### EXAMPLE 1A

### Phenyl[(trimethylsilyl)oxy]acetonitrile

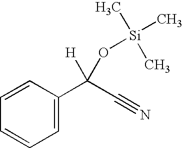

5.63 g (55 mmol) of trimethylsilyl cyanide and 5.31 g (50 mmol) of benzaldehyde give 8.80 g (86% of theory) of product.

HPLC (method 3): Rₜ=3.38 min

MS (DCI): m/z=223 (M+NH₄)⁺

### EXAMPLE 2A

### 4-Chlorophenyl[(trimethylsilyl)oxy]acetonitrile

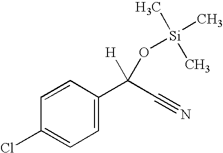

5.63 g (55 mmol) of trimethylsilyl cyanide and 7.03 g (50 mmol) of 4-chlorobenzaldehyde give 10.84 g (90% of theory) of product.

HPLC (method 1): Rₜ=3.96 min

MS (ESIpos): m/z=239 (M+H)⁺

### EXAMPLE 3A

### 4-Methoxyphenyl[(trimethylsilyl)oxy]acetonitrile

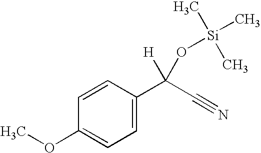

5.63 g (55 mmol) of trimethylsilyl cyanide and 6.81 g (50 mmol) of 4-methoxybenzaldehyde give 10.23 g (87% of theory) of product.

HPLC (method 3): Rₜ=3.56 min

MS (El): m/z=235 (M)⁺

General Procedure [B]:

Reaction of TMS-cyanohydrins with methyl 3-methyl-2-butenoate

1 eq. of the TMS-cyanohydrin in question is, in a 250 ml three-necked flask which was thoroughly dried by heating, dissolved under argon in absolute diethyl ether, and the resulting solution is cooled to −78° C. 1.05 eq. of 2 M LDA solution in THF/heptane/ethylbenzene are added dropwise over a period of 30 min. The mixture is stirred at this temperature for another 30 min, and 1 eq. of methyl 3-methyl-2-butenoate, dissolved in a little absolute diethyl ether, is then added dropwise. Over a period of 5 hours, the mixture is allowed to warm to 0° C.-10° C. Saturated ammonium chloride solution is then added, and the mixture is stirred for 10 min. The phases are separated and the ethereal phase is washed two more times with saturated ammonium chloride solution. After drying over magnesium sulfate and filtration, the solvent is removed on a rotary evaporator, giving the product which is used without further purification for the next synthesis step.

The following compounds were prepared according to this procedure:

### EXAMPLE 4A

### Methyl 4-cyano-3,3-dimethyl-4-phenyl-4-[(trimethylsilyl)oxy]butanoate

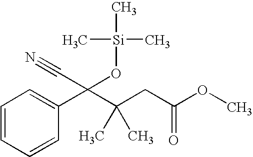

8.80 g (43 mmol) of phenyl[(trimethylsilyl)oxy]acetonitrile, after deprotonation with 22.5 ml of 2 M LDA solution, and 5.04 g (43 mmol) of methyl 3-methyl-2-butenoate give 13.69 g (67% of theory) of the title compound as crude product.

HPLC (method 3): Rₜ=5.53 min

MS (DCI): m/z=337 (M+NH₄)⁺

### EXAMPLE 5A

### Methyl 4-(4-chlorophenyl)-4-cyano-3,3-dimethyl-4-[(trimethylsilyl)oxy]butanoate

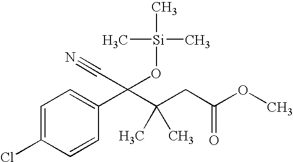

4.80 g (20 mmol) of 4-chlorophenyl[(trimethylsilyl)oxy]acetonitrile, after deprotonation with 10.5 ml of 2 M LDA solution, and 2.28 g (20 mmol) of methyl 3-methyl-2-butenoate give 7.48 g (76% of theory) of the title compound as crude product.

HPLC (method 3): Rₜ=5.64 min

MS (DCI): m/z=371 (M+NH₄)⁺

### EXAMPLE 6A

### Methyl 4-cyano-4-(4-methoxyphenyl)-3,3-dimethyl-4-[(trimethylsilyl)oxy]butanoate

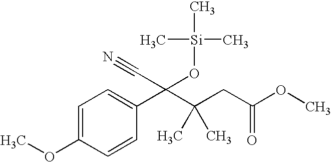

10.23 g (43.5 mmol) of 4-methoxyhphenyl[(trimethylsilyl)oxy]acetonitrile, after deprotonation with 22.8 ml of 2 M LDA solution, and 4.96 g (43.5 mmol) of methyl 3-methyl-2-butenoate give 14.60 g (96% of theory) of the title compound as crude product.

MS (DCI): m/z=367 (M+NH₄)⁺

General procedure [C]:

Desilylation with TBAF

Under an atmosphere of argon, 1 eq. of the methyl butanoate derivatives is dissolved in absolute THF (0.25 M) and cooled to 0° C. At this temperature, 1.1 eq. of a 1 M TBAF solution in THF are slowly added dropwise. The mixture is stirred for another 3 hours, water is then added and the mixture is extracted 3× with dichloromethane. After drying over magnesium sulfate, filtration and removal of the solvent, the product is purified by column chromatography (silica gel: mobile phase cyclohexane/ethyl acetate=85:15) or by kugelrohr distillation.

The following compounds were prepared according to this procedure:

### EXAMPLE 7A

### Methyl 3,3-dimethyl-4-oxo-4-phenylbutanoate

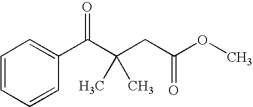

13.44 g (42 mmol) of methyl 4-cyano-3,3-dimethyl-4-phenyl-4-[(trimethylsilyl)oxy]-butanoate and 46.3 ml (46.3 mmol) of a 1 M TBAF solution give 6.54 g (62% of theory) of the title compound as crude product.

HPLC (method 3): Rₜ=4.25 min

MS (DCI): m/z=238 (M+N₄)⁺

Alternative Synthesis Method:

48.4 ml (24.20 mmol; 0.5 M solution in toluene) of potassium hexamethyldisilazide are dissolved in 30 ml of tetrahydrofuran and, at −78° C., 3.26 g (22 mmol) of isobutyrophenone in 10 ml of tetrahydrofuran are added. After 2 hours, 4.04 g (26.40 mmol) of methyl bromoacetate are added. After a further 2 hours, 50 ml of 1N hydrochloric acid are added. The mixture is then extracted with ethyl acetate. The combined organic phases are washed with water and saturated sodium chloride solution and dried with magnesium sulfate, and the solvent is removed. Preparative normal-phase HPLC (column: silica gel, flow rate: 150 ml/min, mobile phase: isohexane/ethyl acetate=9:1) gives the target compound in a yield of 26%.

HPLC (method 3) Rₜ=4.60 min

MS (DCI/NH₃): m/z=238 (M+NH₄)⁺

The following examples are prepared according to the General Procedure [C]:

### EXAMPLE 8A

### Methyl 4-(4-chlorophenyl)-3,3-dimethyl-4-oxobutanoate

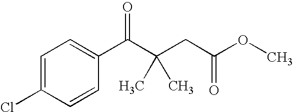

6.15 g (17.4 mmol) of methyl 4-(4-chlorophenyl)-4-cyano-3,3-dimethyl-4-[(trimethylsilyl)oxy]butanoate and 19.1 ml (19.1 mmol) of a 1 M TBAF solution give 4.79 g (90% of theory) of the title compound as crude product.

HPLC (method 3): Rₜ=4.64 min

MS (DCI): m/z=272 (M+NH₄)⁺

### EXAMPLE 9A

### Methyl 4-(4-methoxyphenyl)-3,3-dimethyl-4-oxobutanoate

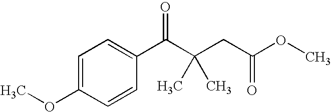

14.48 g (41.4 mmol) of methyl 4-cyano-4-(4-methoxyphenyl)-3,3-dimethyl-4-[(trimethylsilyl)oxy]butanoate and 45.6 ml (45.6 mmol) of a 1 M TBAF solution give 5.45 g (42% of theory) of the title compound as crude product.

HPLC (method 3): Rₜ=4.24 min

MS (ESIpos): m/z=251 (M+H)⁺

General Procedure [D]:

Ester Hydrolysis

The ester to be hydrolyzed is dissolved in a THF/methanol mixture (1:1), and the solution is cooled to 0° C. At this temperature, 2 eq. of 1N aqueous sodium hydroxide solution are slowly added dropwise. After the reaction has ended (the reaction is monitored by TLC), in each case identical portions of a 1N aqueous sodium hydroxide solution and dichloromethane are added. The organic phase is extracted twice with 1N aqueous sodium hydroxide solution. The combined aqueous phases are then acidified with concentrated hydrochloric acid and the product is extracted three times with dichloromethane. Drying over sodium sulfate, filtration and evaporation of the solvent give the product, which is used without further purification for the next synthesis step.

The following compounds were prepared according to this procedure:

### EXAMPLE 10A

### 5-Hydroxy-4,4-dimethyl-5-phenyldihydro-2(3H)-furanone

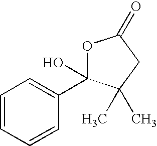

6.52 g (29.6 mmol) of methyl 3,3-dimethyl-4-oxo-4-phenylbutanoate give 5.20 g (83% of theory) of product.

HPLC (method 3): Rₜ=3.88 min

MS (DCI): m/z=224 (M+NH₄⁾⁺

### EXAMPLE 11A

### 5-(4-Chlorophenyl)-5-hydroxy-4,4-dimethyldihydro-2(3H)-furanone

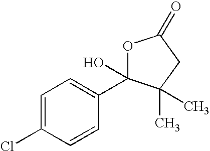

5.11 g (20 mmol) of methyl 4-(4-chlorophenyl)-3,3-dimethyl-4-oxobutanoate give 3.60 g (72% of theory) of product.

HPLC (method 3): Rₜ=4.22 min

MS (DCI): m/z=258 (M+NH₄)⁺

### EXAMPLE 12A

### 5-Hydroxy-5-(4-methoxyphenyl)-4,4-dimethyldihydro-2(3H)-furanone

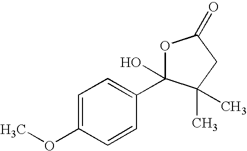

5.22 g (20.9 mmol) of methyl 4-(4-methoxyphenyl)-3,3-dimethyl-4-oxobutanoate give 4.97 g (97% of theory) of product.

HPLC (method 3): Rₜ=3.95 min

MS (ESIpos): m/z=237 (M+H)⁺

General Procedure [E]:

Friedel-Crafts Acylation of Aromatic Compounds with Carboxylic Anhydrides

Under argon, aluminum trichloride (2.4 eq.) is initially charged in a three-necked flask fitted with reflux condenser, and 1,2-dichloroethane (20 ml per g of aluminum trichloride) is added at RT. In an ice-bath, the suspension is cooled to 0° C., and the anhydride (1.05 eq.) is then added a little at a time. After the addition has ended, stirring is continued for 5 min and benzene (1.0 eq.) is then slowly added dropwise. Overnight, the mixture is slowly warmed to room temperature and then poured on ice, and the precipitate is redissolved in 1N hydrochloric acid. The aqueous phase is extracted 2× with dichloromethane, the combined organic phases are washed 2× with water and dried over sodium sulfate and the solvent is removed under reduced pressure. The substance is used as crude product for subsequent reactions.

The following compounds were prepared according to following procedure:

### EXAMPLE 13A

### 2,3-Dimethyl-4-oxo-4-phenylbutanoic acid

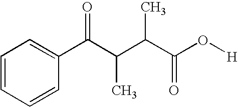

1.4 g of benzene and 2.46 g of 2,3-dimethylbutanedicarboxylic anhydride give 0.3 g (12% of theory) of product.

HPLC (method 6): Rₜ=3.42 min

MS(ESI-POS): m/z=207 (M+H⁺)

### EXAMPLE 14A

### 2,2-Dimethyl-4-oxo-4-phenylbutanoic acid

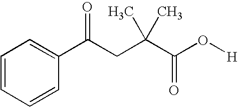

5.8 g of benzene and 10.0 g of 2,2-dimethylbutanedicarboxylic anhydride give 12.6 g, (82% of theory) of product.

m.p.: 174° C.

HPLC (method 3): Rₜ=3.91 min

MS (DCI): m/z=207 (M+H⁺), 224 (M+NH₄)⁺

### EXAMPLE 15A

### cis-2-Benzoylcyclohexanecarboxylic acid

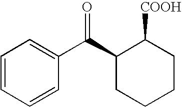

10.0 g of benzene and 20.7 g of cis-cyclohexane-1,2-carboxylic anhydride give 19.7 g (60% of theory) of product as a mixture of enantiomers.

HPLC (method 8): Rₜ=2.89 min

MS (ESI-POS): m/z=233 (M+H⁺)

### EXAMPLE 16A

### trans-2-Benzoylcyclohexanecarboxylic acid

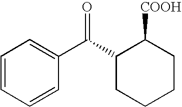

4.8 g of benzene and 10.0 g of trans-cyclohexane-1,2-carboxylic anhydride give 3.75 g (45.5% of theory) as a mixture of enantiomers.

HPLC (method 8): Rₜ=2.91 min

MS (ESI-POS): m/z=233 (M+H⁺)

### EXAMPLE 17A

### Ethyl 3,3-dimethyl-4-phenyl-4-oxobutanoate

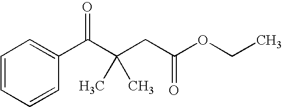

38.20 g (489.55 mmol) of sodium amide are initially charged in 300 ml of toluene, and 32.98 g (222.52 mmol) of isobutyrophenone in 50 ml of toluene are added dropwise at room temperature. After 3 h, 50.00 g (233.65 mmol) of ethyl iodoacetate in 50 ml of toluene are added dropwise at 0° C., and the mixture is stirred for another 2 h. 500 ml of ice-water are then added, and the mixture is extracted with ethyl acetate. The organic phase is washed with saturated sodium chloride solution. After drying over magnesium sulfate, the solution is concentrated and the residue is distilled at 152-158° C. (10 mbar). This gives 36.7 g (70%) of the target compound.

LCMS (method 10): Rₜ=2.93 min

MS (ESIpos): m/z=235 (M+H)⁺

### EXAMPLE 18A

### 3,3-Dimethyl-4-oxo-4-phenylbutanoic acid and 5-hydroxy-4,4-dimethyl-5-phenyl-dihydro-2(3H)-furanone

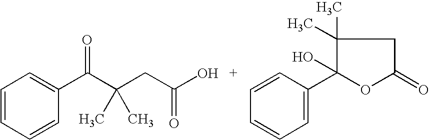

12 g (51.22 mmol) of ethyl 3,3-dimethyl-4-phenyl-4-oxobutanoate are initially charged in 60 ml of tetrahydrofuran and 60 ml of methanol and, at room temperature, stirred with 4.10 g (102.44 mmol) of aqueous sodium hydroxide solution for 2 hours. The solvent is distilled off, and the residue is then acidified with 2N hydrochloric acid. The product is filtered off with suction and recrystallized from water/ethanol.

This gives 8.90 g (84%) of the target compound as a mixture.

LC-MS (method 10): Rₜ=2.31 min

MS (ESIpos): m/z=207 (M+H)⁺

General Procedure [F]:

Nitration of Aromatic Compounds

Under argon, fuming nitric acid is cooled in a flask to −15° C., and at this temperature, the aromatic compound is added a little at a time (300 mg per 1 ml of nitric acid) in an argon countercurrent. After 30 min, the mixture is poured onto ice and extracted 2× with dichloromethane. The combined organic phases are washed 2× with water and dried over sodium sulfate, and the solvent is removed under reduced pressure. The product is obtained as a mixture of the m-isomer with the p-isomer and/or the o-isomer and is reacted further without further purification.

The following compounds are prepared according to this procedure:

### EXAMPLE 19A

### 2,3-Dimethyl-4-(3-nitrophenyl)-4-oxobutanoic acid

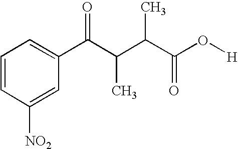

300 mg of 2,3-dimethyl-4-(phenyl)-4-oxobutanoic acid give 280 mg of crude product.

HPLC (method 8): Rₜ=2.43 min, 2.46 min (mixture of diastereomers)

MS (ESI-POS): m/z=252 (M+H⁺)

### EXAMPLE 20A

### 2,2-Dimethyl-4-(3-nitrophenyl)-4-oxobutanoic acid

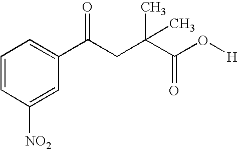

5.0 g of 2,2-dimethyl-4-(phenyl)-4-oxobutanoic acid give 5.1 g (83.9% of theory) of product.

HPLC (method 3): Rₜ=3.95 min

MS (DCI): m/z=252 (M+H⁺), 269 (M+NH₄)⁺

### EXAMPLE 21A

### cis-2-(3-Nitrobenzoyl)cyclohexanecarboxylic acid

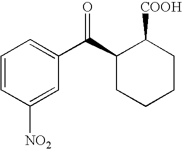

5.0 g of the cis 2-benzoylcyclohexanecarboxylic acid give 5.0 g of crude product as a mixture of enantiomers.

HPLC (method 6): Rₜ=3.66 min, 3.74 min (m- and p-product)

MS (ESI-POS): m/z=278 (M+H⁺)

### EXAMPLE 22A

### trans-2-(3-Nitrobenzoyl)cyclohexanecarboxylic acid

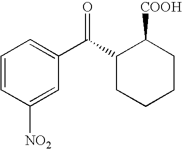

3.75 g of trans-2-benzoylcyclohexanecarboxylic acid give 3.5 g of crude product as a mixture of enantiomers.

HPLC (method 8): Rₜ=2.54 min, 2.62 min (m- and p-product)

MS (ESI-POS): m/z=278 (M+H⁺)

### EXAMPLE 23A

### 5-Hydroxy-4,4-dimethyl-5-(3-nitrophenyl)dihydro-2(3H)-furanone and 3,3-dimethyl-4-(3-nitrophenyl)-4-oxobutanoic acid

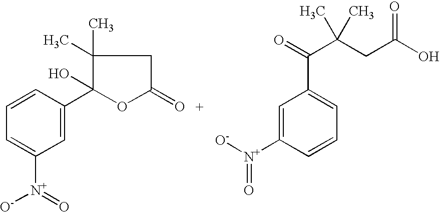

8.65 g (41.94 mmol) of a mixture of 3,3-dimethyl-4-oxo-4-phenylbutanoic acid and 5-hydroxy-4,4-dimethyl-5-phenyldihydro-2(3H)-furanone are initially charged in 20 ml of sulfuric acid, and 5.49 g of nitric acid (65% strength) in 10 ml of sulfuric acid are added at −15° C. Stirring is continued at 0° C. for 1 hour. Ice-water is then added, and the mixture is extracted with ethyl acetate. The combined organic phases are washed with water and dried over magnesium sulfate. After removal of the solvent, the product mixture crystallizes out. The yield is 10.5 g (quantitative).

LC-MS (method 6): Rₜ=3.40/3.50 min

MS (ESIneg): m/z=250 (M−H)⁻

### EXAMPLE 24A

### 5-Hydroxy-4,4-dimethyl-5-(3-nitrophenyl)dihydro-2(3H)-furanone and 5-hydroxy-4,4-dimethyl-5-(4-nitrophenyl)dihydro-2(3H)-furanone

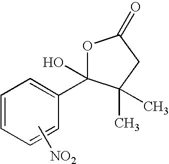

In a flask, fuming nitric acid (12 ml) is, under argon, cooled to −15° C. At this temperature, 5 g (24.5 mmol) of 5-hydroxy-4,4-dimethyl-5-phenyldihydro-2(3H)-furanone are added a little at a time as a solid. Stirring is continued at −15° C. for half an hour and the mixture is then poured onto ice and extracted three times with dichloromethane. The combined extracts are dried over magnesium sulfate. Purification is carried out by column chromatography (dichloromethane/methanol 97:3). This gives 6.23 g of a product mixture of the title compounds as crude product.

HPLC (method 3): Rₜ=4.06 min

MS (DCI): m/z=269 (M+NH₄)⁺

### EXAMPLE 25A

### 5-(4-Chloro-3-nitrophenyl)-5-hydroxy-4,4-dimethyldihydro-2(3H)-furanone

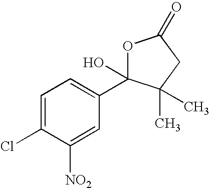

In a flask, fuming nitric acid (7 ml) is, under argon, cooled to −15° C. At this temperature, 3.42 g (14.2 mmol) of 5-(4-chlorophenyl)-5-hydroxy-4,4-dimethyldihydro-2(3H)-furanone are added a little at a time as a solid. Stirring at −15° C. is continued for 45 min (until the entire solid has gone into solution) and the mixture is then poured onto ice and extracted three times with dichloromethane. The combined extracts are dried over sodium sulfate. After filtration and removal of the solvent, the residue is dried under high vacuum. This gives 3.89 g (87% of theory, purity 91%) of product.

HPLC (method 3): Rₜ=4.20 min

MS (DCI): m/z=303 (M+NH₄)⁺

### EXAMPLE 26A

### 5-Hydroxy-5-(4-methoxy-3-nitrophenyl)-4,4-dimethyldihydro-2(3H)-furanone

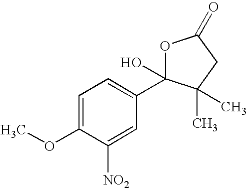

Since 5-hydroxy-5-(4-methoxyphenyl)-4,4-dimethyldihydro-2(3H)-furanone is not obtained as a solid but as a relatively viscous oil, the fuming nitric acid is, at −15° C., slowly added to the substance. After a short period of time, a vigorous reaction (evolution of nitrous gases) sets in. Stirring is continued for 30 min and the mixture is then poured onto ice and extracted three times with dichloromethane. This gives 5.65 g of the title compound as a crude product. The crude product is, without further work-up, used for subsequent reactions.

MS (DCI): m/z=299 (M+NH₄)⁺

General Procedure [G]:

Cyclization to the Pyridazinone and Simultaneous Reduction of a Nitro Group

The nitro compound (1.0 eq.) and hydrazine monohydrate (20.0 eq.) are, at RT, initially charged in ethanol (0.1 M solution), 10% by weight of palladium/carbon (10% by weight) are then added and the mixture is heated under reflux overnight. The catalyst is then filtered off and washed with ethanol and the solvent is subsequently removed under reduced pressure. The product is purified by repeated recrystallization from ethanol or by column chromatography (silica gel for removing the o-isomer, preparative HPLC (method 12) for removing the p-isomer).

The following compounds were prepared according to this procedure:

### EXAMPLE 27A

### 6-(3-Aminophenyl)-4,5-dimethyl-4,5-dihydro-3(2H)-pyridazinone

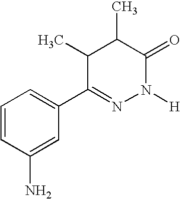

500 mg of 2,3-dimethyl-4-(3-nitrophenyl)-4-oxobutanoic acid give 210 mg of crude product which is reacted further without further purification.

HPLC (method 6): Rₜ=0.85 min; 1.10 min (mixture of diastereomers)

MS (ESI-POS): m/z=218 (M+H⁺)

### EXAMPLE 28A

### 6-(3-Aminophenyl)-4,4-dimethyl-4,5-dihydro-3 (2H)-pyridazinone

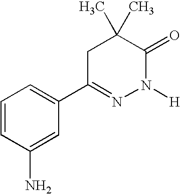

4.95 g of 2,2-dimethyl-4-(3-nitrophenyl)-4-oxobutanoic acid give 1.24 g (28% of theory) of product.

HPLC (method 3): Rₜ=2.90 min

MS (ESI-POS): m/z=218 (M+H⁺)

During chromatography, the o-isomer could be isolated as a by-product (HPLC method 3: Rₜ=3.10 min)

### EXAMPLE 29A

### 4-(3-Aminophenyl)-cis-4a,5,6,7,8,8a-hexahydro-1(2H)-phthalazinone

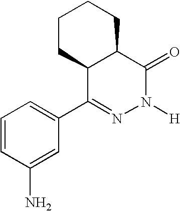

500 mg of cis-2-(3-nitrobenzoyl)cyclohexanecarboxylic acid give, in quantitative yield, the target compound as crude product which is reacted without further purification.

HPLC (method 6): Rₜ=2.32 min, 2.55 min, 3.34 min, 3.40 min

MS (ESI-POS): all m/z=244 (M+H⁺)

### EXAMPLE 30A

### 4-(3-Aminophenyl)-trans-4a,5,6,7,8,8a-hexahydro-1(2H)-phthalazinone

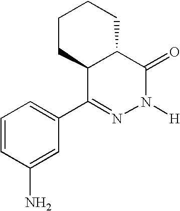

3.5 g of crude product trans-2-(3-nitrobenzoyl)cyclohexanecarboxylic acid give, in quantitative yield, the target compound as crude product which is reacted without further pufication.

HPLC (method WTB): Rₜ=1.91 min

### EXAMPLE 31A

### 6-(3-Aminophenyl)-5,5-dimethyl-4,5-dihydro-3(2H)-pyridazinone and 6-(4-amino-phenyl)-5,5-dimethyl-4,5-dihydro-3(2H)-pyridazinone

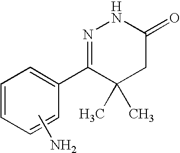

2.98 g (11.9 mmol) of a mixture of 5-hydroxy-4,4-dimethyl-5-(3-nitrophenyl)dihydro-2(3H)-furanone and 5-hydroxy-4,4-dimethyl-5-(4-nitrophenyl)-dihydro-2(3H)-furanone are dissolved in 40 ml of ethanol at RT, and 8.91 g (178 mmol) of hydrazine monohydrate are added. This is followed by the addition of 300 mg of palladium/carbon (10% by weight), and the reaction mixture is heated at reflux for 20 hours. The hot reaction mixture is then filtered through Celite, the filter cake is washed with hot ethanol and the filtrate is concentrated to dryness. The residue is crystallized from ethanol. This gives 1.09 g (34% of theory) of a product mixture comprising 80% of meta- and 20% of para-product. Recrystallization from the mother liquor gives 1.03 g (30% of theory) of a product mixture comprising 74% of para- and 26% of meta-product. The two fractions are combined and para- and meta-product are separated by preparative HPLC (method 12).

HPLC (method 3): Rₜ=2.53 min (para) and 2.83 min (meta)

MS (EI): m/z=217 (M)⁺

### EXAMPLE 32A

### 6-(3-Amino-4-chlorophenyl)-5,5-dimethyl-4,5-dihydro-3(2H)-pyridazinone

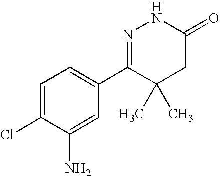

3.86 g (13.5 mmol) of 5-(4-chloro-3-nitrophenyl)-5-hydroxy-4,4-dimethyldihydro-2(3H)-furanone are dissolved in 40 ml of ethanol at RT, and 10.14 g (203 mmol) of hydrazine monohydrate are added. This is followed by the addition of 350 mg of palladium/carbon (10%), and the reaction mixture is heated at reflux for 24 hours. The hot reaction mixture is then filtered through Celite, the filter cake is washed with hot ethanol and the filtrate is concentrated to dryness. The residue is crystallized from ethanol. This gives 1.66 g (48% of theory) of product. Crystallization from the mother liquor gives another 0.34 g (10% of theory) of product.

HPLC (method 3): Rₜ=3.56 min

MS (DCI): m/z=269 (M+NH₄)⁺

### EXAMPLE 33A

### 6-(4-Methoxy-3-nitrophenyl)-5,5-dimethyl-4,5-dihydro-3-(2H)-pyridazinone

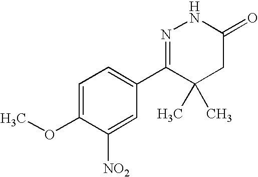

5.40 g (19.2 mmol) of 5-hydroxy-5-(4-methoxy-3-nitrophenyl)-4,4-dimethyldihydro-2(3H)-furanone are dissolved in 60 ml of absolute ethanol at RT, 3.84 g (77 mmol) of hydrazine monohydrate are added and the mixture is heated at reflux for 6 hours. Removal of the solvent results in the precipitation of a brown solid which is filtered off and dried. Drying under high vacuum gives 1.80 g (34% of theory) of product.

HPLC (method 3): Rₜ=3.80 min

MS (DCI): m/z=295 (M+NH₄)⁺

### EXAMPLE 34A

### 6-(3-Amino-4-methoxyphenyl)-5,5-dimethyl-4,5-dihydro-3-(2H)-pyridazinone

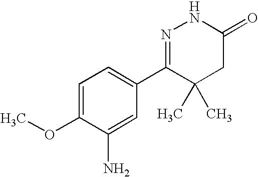

1.77 g (6.4 mmol) of 6-(4-methoxy-3-nitrophenyl)-5,5-dimethyl-4,5-dihydro-3-(2H)-pyridazinone are dissolved in 50 ml of absolute ethanol at RT, and 3.20 g (64 mmol) of hydrazine monohydrate and 170 mg of palladium/carbon (10% by weight) are added. The reaction mixture is heated at reflux for 10 hours and then concentrated and purified by column chromatography (ethyl acetate/cyclohexane 7:3). This gives 0.7 g (24% of theory) of the title compound.

HPLC (method 3): Rₜ=2.91 min

MS (EI): m/z=247 (M)⁺

### EXAMPLE 35A

### 6-(3-Amino-4-methylphenyl)-5-ethyl-5-methyl-4,5-dihydro-3(2H)-pyridazinone

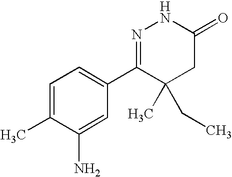

Prepared analogously to Example 36A from 15.00 g (53.71 mmol) of 5-ethyl-5-methyl-6-(4-methyl-3-nitrophenyl)-4,5-dihydro-3(2H)-pyridazinone and 26.89 g (537.08 mmol) of hydrazine hydrate in a yield of 82%.

¹H-NMR (200 MHz, DMSO): δ=0.72 (t, 3H), 1.12 (s, 3H), 1.35-1.80 (m, 2H), 2.05 (s, 3H), 2.30 (q, 2H), 3.33 (s, 1H), 4.89 (br.s, 2H), 6.50 (dd, 1H), 6.68 (d, 1H), 6.88 (d, 1H), 10.83 (br.s, 1H).

### EXAMPLE 36A

### 5-Ethyl-6-(3-isocyanato-4-methylphenyl)-5-methyl-4,5-dihydro-3(2H)-pyridazinone

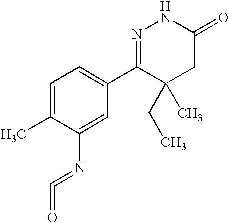

4.91 g (20 mmol) of 6-(3-amino-4-methylphenyl)-5-ethyl-5-methyl-4,5-dihydro-3(2H)-pyridazinone are initially charged in 200 ml of dichloromethane and, at 0° C., initially 8.57 g (40 mmol) of 1,8-bis-(dimethylamino)naphthalene and then 2.85 g (14.40 mmol) of trichloromethyl chloroformate in 80 ml of dichloromethane are added. After 1 hour at room temperature, the mixture is diluted with 50 ml of dichloromethane. The organic phase is washed with ice-water, 1N hydrochloric acid and saturated sodium chloride solution and dried over magnesium sulfate. After concentration of the solution and trituration of the residue with n-heptane, the target compound is isolated in a yield of 5.30 g (98%).

MS (DCI/NH₃): m/z=272 (M+H⁺)

### EXAMPLE 37A

### (4-Bromophenyl)-[(trimethylsilyl)oxy]acetonitrile

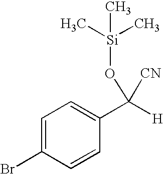

According to a procedure of K. Deuchert, U. Hertenstein, S. Hünig, G. Wehner, Chem. Ber. 1979, 112, 2045-2061, under an atmosphere of argon, a small spatula tip of zinc iodide is added as catalyst to 2.73 g (27.5 mmol) of trimethylsilyl cyanide, and the mixture is heated to 60° C. At this temperature, 4.23 g (25 mmol) of 4-bromo-benzaldehyde are added a little at a time as a solid. The temperature is then increased to 95° C. and maintained at this level for 8 hours. Purification is then carried out by kugelrohr distillation under high vacuum (220-230° C.). This gives 6.11 g (85% of theory) of product as a yellow oil.

HPLC (method 3): Rₜ=3.88 min

MS (ESIpos): m/z=306 (M+Na)⁺

### EXAMPLE 38A

### Methyl 4-(4-bromophenyl)-4-cyano-3,3-dimethyl-4-[trimethylsilyl)oxy]butanoate

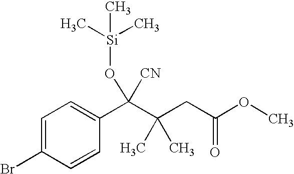

According to a procedure from S. Hünig, G. Wehner, Chem. Ber. 1980, 113, 302-323, 6 g (21.11 mmol) of (4-bromophenyl)-[(trimethylsilyl)oxy]acetonitrile are, under an atmosphere of argon, dissolved in 21 ml of dry diethyl ether, and the resulting solution is cooled to −78° C. Over a period of 20 min, 11.1 ml (22.2 mmol) of a 2-molar solution of lithium diisopropylamide are added dropwise. After 30 min, a solution of 2.48 g (21.11 mmol) of methyl 3-methyl-2-butenoate in 2 ml of dry diethyl ether is added dropwise. In a cooling bath, the reaction mixture is, over a period of 4-5 hours, slowly allowed to warm to room temperature. 40 ml of saturated ammonium chloride solution are then added, and stirring is continued at room temperature for 10 min. The phases are separated and the organic phase is dried over magnesium sulfate. Filtration and evaporation of the solvent give 9.48 g of crude product which is used without further purification for the next synthesis.

HPLC (method 1): Rₜ=5.72 min

MS (EI): m/z=397 (M)⁺

### EXAMPLE 39A

### Methyl 4-(4-bromophenyl)-3,3-dimethyl-4-oxobutanoate

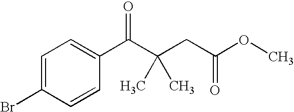

Under an atmosphere of argon, 8.65 g (crude product) of methyl 4-(4-bromophenyl)-4-cyano-3,3-dimethyl-4-[trimethylsilyl)oxy]butanoate are dissolved in 87 ml of dry tetrahydrofuran, and the solution is cooled in an ice-bath to 0° C. At this temperature, 21.7 ml (21.7 mmol) of a 1-molar solution of tetrabutylammonium fluoride are slowly added dropwise. After 4.5 hours at 0° C., 75 ml of water are added and the mixture is extracted 3 times with dichloromethane. The combined organic phases are dried over magnesium sulfate. After filtration and evaporation of the solvent, purification is carried out by column chromatography (silica gel: cyclohexane/ethyl acetate 85:15), giving 4.25 g (57% of theory over 2 steps) of product.

HPLC (method 3): Rₜ=4.84 min

MS (EI): m/z=298 (M)⁺

### EXAMPLE 40A

### Methyl 4-(4-bromo-3-nitrophenyl)-3,3-dimethyl-4-oxobutanoate

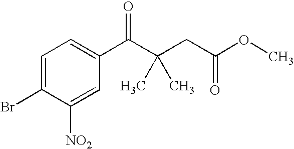

Under argon, 6 ml of fuming nitric acid are cooled to −30° C., and 3.71 g (12.4 mmol) of methyl 4-(4-bromophenyl)-3,3-dimethyl-4-oxobutanoate are added dropwise at this temperature. Stirring at −30° C. is continued for 1 hour and the mixture is then poured onto ice and extracted 3 times with dichloromethane. The combined organic extracts are dried over magnesium sulfate. After filtration and evaporation of the solvent, the product is purified by column chromatography (silica gel: cyclohexane/ethyl acetate 9:1). This gives 2.83 g (66% of theory) of product as a yellow oil.

HPLC (method 3): Rₜ=4.75 min

MS (DCI): m/z=361 (M+NH₄)⁺

### EXAMPLE 41A

### Methyl 4-(3-aminophenyl)-3,3-dimethyl-4-oxobutanoate

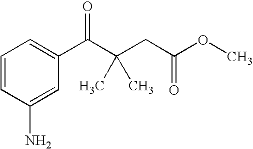

0.85 g (2.46 mmol) of methyl 4-(4-bromo-3-nitrophenyl)-3,3-dimethyl-4-oxobutanoate is dissolved in 12 ml of degassed ethanol, 131 mg of palladium on carbon (10%) are added and the mixture is stirred under an atmosphere of hydrogen until reaction monitoring by analytical HPLC shows complete conversion. The mixture is filtered through kieselguhr and the solvent is evaporated. The residue is taken up in ethyl acetate and washed with saturated sodium bicarbonate solution. The organic phase is dried over magnesium sulfate. Filtration and concentration of the solvent give 0.52 g (83% of theory) of product which is used as crude product for further syntheses.

### EXAMPLE 42A

### 4-(2-Hydroxy-5-nitrophenyl)-3,3-dimethyl-4-oxobutanoic acid

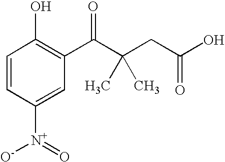

10.00 g (37.14 mmol) of 4-(2-fluoro-5-nitrophenyl)-3,3-dimethyl-4-oxobutanoic acid and 7.02 g (83.57 mmol) of sodium bicarbonate are initially charged in 100 ml of water, 11.46 g (81.72 mmol) of ammonium hydroxide are added and the mixture is stirred under reflux overnight. After cooling, the mixture is acidified with 1N hydrochloric acid and extracted with ethyl acetate, and the organic phase is washed with water. Drying over magnesium sulfate and removal of the solvent by distillation is followed by recrystallization from methylene chloride/n-pentane and drying under reduced pressure.

This gives 6.52 g (66% of theory) of product.

HPLC (method 3): Rₜ=3.90 min

MS (DCI/NH₃): m/z=285 (M+NH₄)⁺.

### EXAMPLE 43A

### 4-(2-Cyano-5-nitrophenyl)-3,3-dimethyl-4-oxobutanoic acid

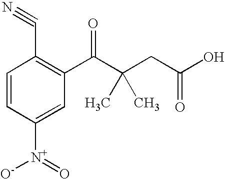

The preparation of 4-(2-cyano-5-nitrophenyl)-3,3-dimethyl-4-oxobutanoic acid is carried out using the starting material 4-(2-fluoro-5-nitrophenyl)-3,3-dimethyl-4-oxobutanoic acid, similarly to the literature references Heterocycles 1987, 26, 1227 and Synth. Commun. 1985, 15, 479.

### EXAMPLE 44A

### 6-(2-Hydroxy-5-nitrophenyl)-5,5-dimethyl-4,5-dihydro-3(2H)-pyridazinone

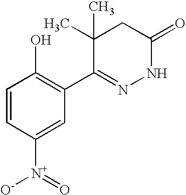

26.00 g (94.12 mmol) of 4-(2-cyano-5-nitrophenyl)-3,3-dimethyl-4-oxobutanoic acid (Example 43A) are dissolved in 400 ml of ethanol, and 47.12 g (941.19 mmol) of hydrazine hydrate are added dropwise under reflux. The mixture is stirred at boiling point for 5 h and the solution is then concentrated to 100 ml. Water is added to the residue and the volume is concentrated to 200 ml. The crystals are then filtered off with suction and washed with water and diethyl ether. Drying under reduced pressure gives 20.03 g (81% of theory) of product.

HPLC (method 3): Rₜ=3.50 min

MS (DCI/NH₃): m/z=281 (M+NH₄)⁺

Alternatively, using the same synthesis procedure, Example 44A can also be prepared from 4-(2-hydroxy-5-nitrophenyl)-3,3-dimethyl-4-oxobutanoic acid (Example 42A).

### EXAMPLE 45A

### 6-(5-Amino-2-hydroxyphenyl)-5,5-dimethyl-4,5-dihydro-3(2H)-pyridazinone

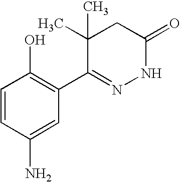

3.00 g (11.40 mmol) of 6-(2-hydroxy-5-nitrophenyl)-5,5-dimethyl-4,5-dihydro-3(2H)-pyridazinone (Example 45A) are dissolved in 150 ml of ethanol, and 0.30 g palladium/carbon (10%) is added. At boiling point, 5.70 g (113.96 mmol) of hydrazine hydrate are added dropwise. After 18 h of stirring under reflux, the solvent is removed and the oily residue is crystallized from diethyl ether. The crystals are triturated with water and filtered off with suction. After washing with diethyl ether, the crystals are dried under reduced pressure. This gives 1.84 g (69% of theory) of product.

HPLC (method 3): Rₜ=2.30 min

MS (ESI pos): m/z=234 (M+H)⁺

### PREPARATION EXAMPLES

### EXAMPLE 1

### N-(2,4-Difluorophenyl)-N′-[3-(4,4-dimethyl-6-oxo-1,4,5,6-tetrahydro-3-pyridazinyl)-phenyl]urea

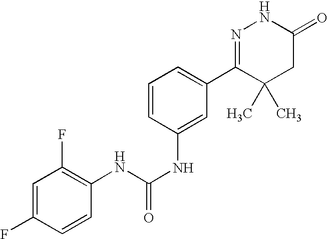

At room temperature, 2 ml of abs. THF are added to 50 mg (0.23 mmol) of 6-(3-aminophenyl)-5,5-dimethyl-4,5-dihydro-3(2H)-pyridazinone, and 71.4 mg (0.46 mmol) of 2,4-difluorophenyl isocyanate are then added. Initially, the 6-(3-aminophenyl)-5,5-dimethyl-4,5-dihydro-3(2H)-pyridazinone does not dissolve completely. Only after the addition of the isocyanate is, after a short period of time, a clear yellow solution formed; however, this solution quickly yields a white precipitate. Stirring is continued overnight and the precipitate is then filtered off. The precipitate is washed with diethyl ether and the white solid is dried under reduced pressure. This gives 46.4 mg (54% of theory) of product.

m.p.: 213° C.

¹H-NMR (200 MHz, DMSO): δ=1.16 (s, 6H), 2.35 (s, 2H), 6.97-7.11 (m, 2H), 7.25-7.39 (m, 3H), 7.65 (s, 1H), 7.99-8.17 (m, 1H), 8.50 (s, br 1H), 9.12 (s, br 1 H), 10.99 (s, 1H).

HPLC (method 3): Rₜ=4.12 min

MS (ESIpos): m/z=373 (M+H)⁺

### EXAMPLE 2

### N-(3-Chloro-4-fluorophenyl)-N′-[3-(4,4-dimethyl-6-oxo-1,4,5,6-tetrahydro-3-pyridazinyl)phenyl]urea

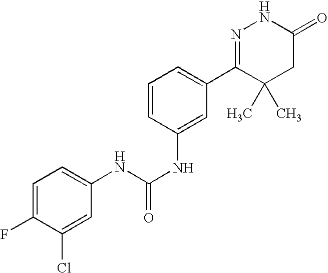

1 ml of abs. THF is added to 30 mg (0.14 mmol) of 6-(3-aminophenyl)-5,5-dimethyl-4,5-dihydro-3(2H)-pyridazinone and 47.4 mg (0.28 mmol) of 3-chloro-4-fluoro-phenyl isocyanate (slightly turbid solution), and the mixture is stirred at room temperature overnight. Formation of a white precipitate is observed.

Work-up: In each case 1 ml of dichloromethane and diethyl ether are added to the reaction mixture, and the white precipitate is filtered off. This gives 40.2 mg (75% of theory) of product.

¹H-NMR (200 MHz, DMSO): δ=1.16 (s, 6H), 2.35 (s, 2H), 7.04 (d, 1H), 7.25-7.42 (m, 4H), 7.63 (s, 1H), 7.76-7.83 (m, 1H), 8.89 (s, 2H), 10.99 (s, 1H).

HPLC (method 3): Rₜ=4.32 min

MS (ESIpos): m/z=389 (M+H)⁺

### EXAMPLE 3

### N-(3-Bromophenyl)-N′-[3-(4,4-dimethyl-6-oxo-1,4,5,6-tetrahydro-3-pyridazinyl)-phenyl]urea

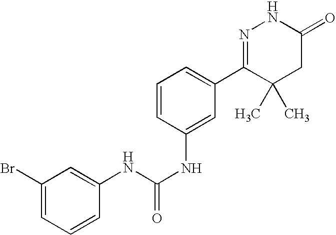

30 mg (0.14 mmol) of 6-(3-aminophenyl)-5,5-dimethyl-4,5-dihydro-3(2H)-pyridazinone are dissolved in 1 ml of absolute THF. 54.7 mg (0.28 mmol) of 3-bromophenyl isocyanate are added, and the mixture is then stirred at room temperature overnight. This results in the precipitation of a white solid. Filtration gives 48.4 mg (84% of theory) of product as a white solid.

m.p.: 207° C.

¹H-NMR (200 MHz, DMSO): δ=1.16 (s, 6H), 2.35 (s, 2H), 6.99-7.45 (m, 6H), 7.64 (s, 1H), 7.85 (s, 1H), 8.90 (s, br 2H), 10.99 (s, br 1H).

HPLC (method 3): Rₜ=4.32 min

MS (ESIpos): m/z=415 (M+H)⁺

### EXAMPLE 4

### N-(4-Chloro-2-methylphenyl)-N′-[3-(4,4-dimethyl-6-oxo-1,4,5,6-tetrahydro-3-pyridazinyl)phenyl]urea

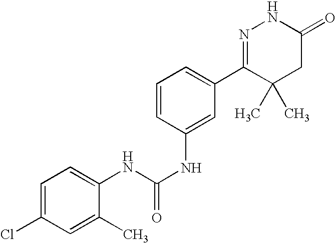

46.3 mg (0.28 mmol) of 4-chloro-2-methylphenyl isocyanate are initially charged in 1 ml of ethyl acetate (not completely soluble), 30 mg (0.14 mmol) of 6-(3-aminophenyl)-5,5-dimethyl-4,5-dihydro-3(2H)-pyridazinone are added and the mixture is stirred at room temperature overnight. The formation of a white precipitate is observed.

Work-up: The precipitate is filtered off, washed with diethyl ether and dried under reduced pressure. Since the product is still showing impurities, it is purified by RP-HPLC. This gives 25.6 mg (48% of theory) of product as a white solid.

m.p.: 232° C.

¹H-NMR (200 MHz, DMSO): δ=1.16 (s, 6H), 2.24 (s, 3H), 2.35 (s, 2H), 6.99-7.06 (m, 1H), 7.15-7.40 (m, 4H), 7.66 (s, 1H), 7.87 (d, 1H), 7.99 (s, 1H), 9.16 (s, 1H), 10.99 (s, 1H).

HPLC (method 3): Rₜ=4.30 min

MS (ESIpos): m/z=385 (M+H)⁺

### EXAMPLE 5

### N-(2,4-Difluorophenyl)-N′-[2-(5,5-dimethyl-6-oxo-1,4,5,6-tetrahydro-3-pyridazinyl)-phenyl]urea

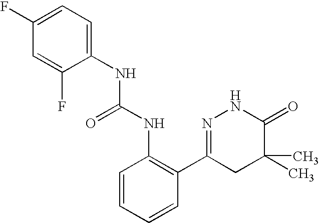

20.3 mg (0.09 mmol) of 6-(2-aminophenyl)-5,5-dimethyl-4,5-dihydro-3(2H)-pyridazinone are suspended in 1 ml of ethyl acetate, and 29 mg (0.18 mmol) of 2,4-difluorophenyl isocyanate are added at room temperature. After a while, the reaction mixture becomes completely clear and the resulting colorless clear solution is stirred overnight. The solvent is removed using a rotary evaporator and the residue is purified by RP-HPLC. This gives 26.1 mg (75% of theory) of product.

¹H-NMR (200 MHz, DMSO): δ=1.10 (s, 6H), 2.83 (s, 2H), 6.98-7.14 (m, 2H), 7.24-7.40 (m, 2H), 7.43-7.50 (m, 1H), 7.82-7.96 (m, 1H), 8.02-8.09 (m, 1H), 9.07 (s, 1H), 9.50 (s, 1H), 10.77 (s, 1H).

HPLC (method 3): Rₜ=4.32 min

MS (ESIpos): m/z=373 (M+H)⁺

### EXAMPLE 6

### N-(3,4-Difluorophenyl)-N′-[3-(5,5-dimethyl-6-oxo-1,4,5,6-tetrahydro-3-pyridazinyl)-phenyl]urea

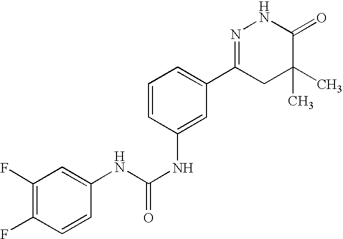

42.8 mg (0.28 mmol) of 3,4-difluorophenyl isocyanate are dissolved in 0.5 ml of ethyl acetate, a solution of 30 mg (0.14 mmol) of 6-(3-aminophenyl)-5,5-dimethyl-4,5-dihydro-3(2H)-pyridazinone in 0.5 ml of ethyl acetate is added and the mixture is stirred at room temperature overnight. The formation of a white precipitate is observed.

Work-up: The reaction mixture is concentrated and the residue is purified by RP-HPLC. This gives 48 mg (93% of theory) of product as a white solid.

m.p.: 200° C.

¹H-NMR (200 MHz, DMSO): δ=1.08 (s, 6H), 2.1 (s, 2H), 7.09-7.18 (m, 1H), 7.26-7.44 (m, 4H), 7.60-7.74 (m, 1H), 7.97 (s, 1H), 8.89 (s, 2H), 10.90 (s, 1H).

HPLC (method 3): Rₜ=4.28 min

MS (ESIpos): m/z=373 (M+H)⁺

General Procedure [H]:

Reaction of Anilines with Isocyanates

Under an atmosphere of inert gas and at room temperature, a solution of 1.0 eq. of aniline in tetrafuran (0.2 M solution) is mixed with 1.2 eq. of isocyanate (dissolved in the same volume of absolute tetrahydrofuran). The reaction mixture is shaken at room temperature overnight. Methylene chloride/diethyl ether (1:1) are added to the reaction mixture, the mixture is shaken for 1 h and the precipitated solid is then obtained by filtration. For purification, the solid is washed with diethyl ether or alternatively purified either by crystallization from methylene chloride or by preparative HPLC.

The following compounds were prepared according to this method:

### EXAMPLE 7

### N-(2,4-Dichlorophenyl)-N′-[3-(6-oxo-1,4,5,6-tetrahydro-3-pyridazinyl)phenyl]urea

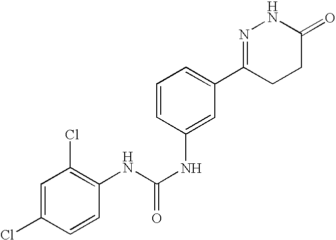

¹H-NMR (200 MHz, DMSO): δ=2.45-2.60 (m, 2H), 2.89-3.03 (m, 2H), 3.07 (s, 1H), 7.15-7.50 (m, 4H), 7.96 (s, 1H), 8.14 (s, 1H), 8.23 (d, 1H), 9.33 (s, 1H), 10.52 (s, 1H).

HPLC (method 3): Rₜ=4.40 min

MS (ESIpos): m/z=377 (M+H)⁺

### EXAMPLE 8

### N-(3,4-Difluorophenyl)-N′-[3-(4-methyl-6-oxo-1,4,5,6-tetrahydro-3-pyridazinyl)phenyl]urea

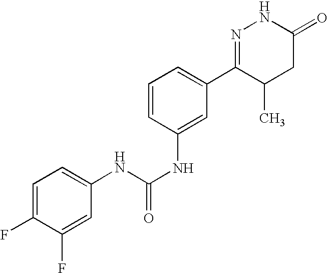

¹H-NMR (200 MHz, DMSO): δ=1.10 (d, 3H), 2.26 (d, 1H), 2.60-2.80 (m, 1H), 3.20-3.45 (m, 1H), 7.10-7.21 (m, 1H), 7.22-7.48 (m, 4H), 7.57-7.73 (m, 1H), 7.99 (s, 1H), 8.89 (s, 2H), 10.99 (s, 1H).

HPLC (method 3): Rₜ=4.10 min

MS (DCI/NH₃): m/z=376 (M+NH₄)⁺

### EXAMPLE 9

### N-(2,4-Difluorophenyl)-N′-[3-(4-methyl-6-oxo-1,4,5,6-tetrahydro-3-pyridazinyl)-phenyl]urea

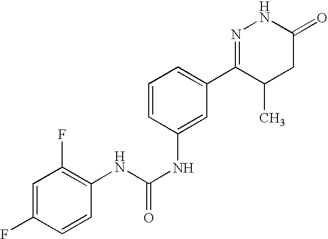 Enantiomer A:

The target compound is prepared as a racemate from the corresponding starting materials and then separated from the other enantiomer using an HPLC method (method 9) specifically developed for this enantiomer separation.

¹H-NMR (200 MHz, DMSO): δ=1.10 (d, 3H), 2.25 (d, 1H), 2.61-2.80 (m, 1H), 3.26-3.42 (m, 1H), 7.00-7.15 (m, 1H), 7.28-7.51 (m, 4H), 7.95-8.20 (m, 2H), 8.49 (s, 1H), 9.15 (s, 1H), 10.99 (s, 1H).

HPLC (method 9): Rₜ=23.63 min

MS (DCI/NH₃): m/z=376 (M+NH₄)⁺

### EXAMPLE 10

### N-(2,4-Difluorophenyl)-N′-[3-(4-methyl-6-oxo-1,4,5,6-tetrahydro-3-pyridazinyl)-phenyl]urea

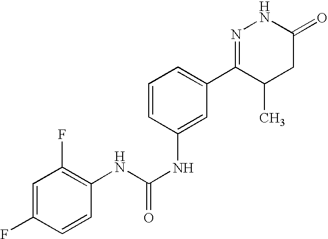 Enantiomer B:

The target compound is prepared as a racemate from the corresponding starting materials and then separated from the other enantiomer using an HPLC method (method 9) specifically developed for this enantiomer separation.

¹H-NMR (200 MHz, DMSO): δ=1.10 (d, 3H), 2.25 (d, 1H), 2.61-2.80 (m, 1H), 3.26-3.42 (m, 1H), 7.00-7.15 (m, 1H), 7.28-7.51 (m, 4H), 7.95-8.20 (m, 2H), 8.49 (s, 1H), 9.15 (s, 1H), 10.99 (s, 1H).

HPLC (method 9): Rₜ=27.13 min

MS (DCI/NH₃): m/z=376 (M+NH₄)⁺

The examples of Table 1 can be obtained by the general procedure [H].

TABLE 1

| | | | MS(ESI+) | HPLC | |
|---|---|---|---|---|---|
| | | | m/z | Rt[min] | HPLC |
| Example No. | Structure | MW | [M + H]⁺ | (%) | method |
| 11 | 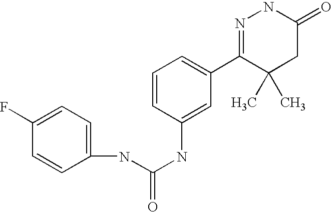 | 354 | 355 | 3.48(100) | 6 |
| 12 | 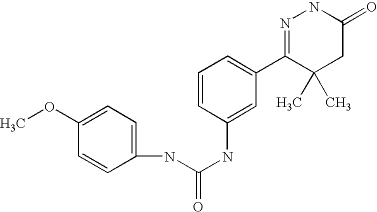 | 366 | 367 | 3.33(100) | 6 |
| 13 | 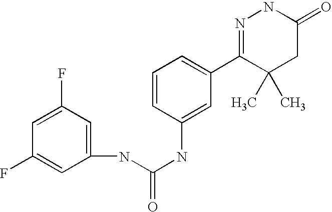 | 372 | 373 | 3.76(100) | 6 |
| 14 | 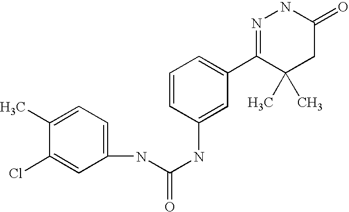 | 385 | 385 | 3.95(100) | 6 |
| 15 | 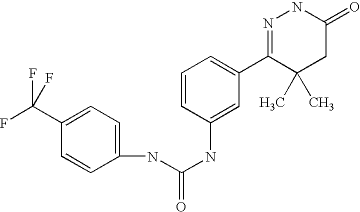 | 404 | 405 | 3.95(100) | 6 |
| 16 | 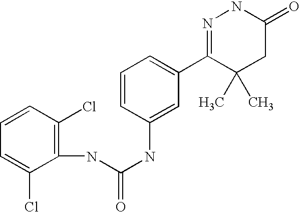 | 405 | 405 | 3.44(100) | 6 |
| 17 | 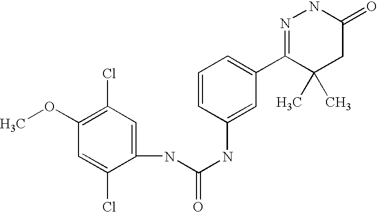 | 435 | 435 | 3.90(100) | 6 |
| 18 | 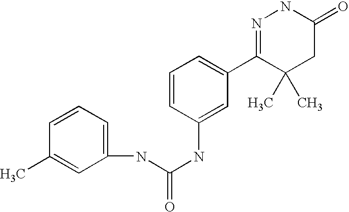 | 350 | 351 | 3.61(100) | 6 |
| 19 | 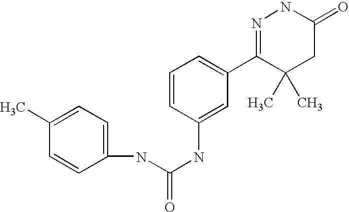 | 350 | 351 | 3.60(100) | 6 |
| 20 | 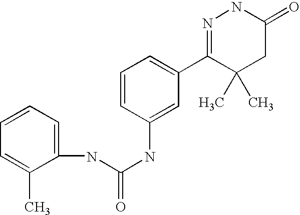 | 350 | 351 | 3.49(100) | 6 |
| 21 | 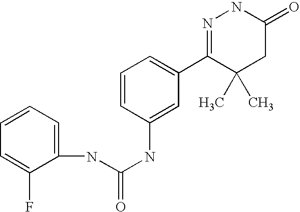 | 354 | 355 | 3.53(100) | 6 |
| 22 | 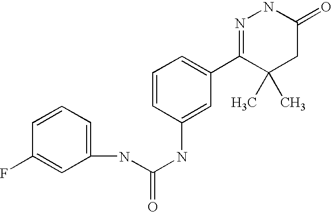 | 354 | 355 | 3.67(92) | 6 |
| 23 | 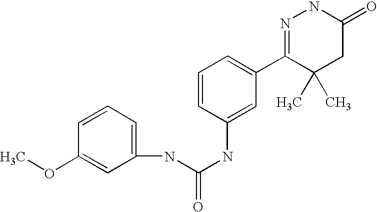 | 366 | 367 | 3.43(100) | 6 |
| 24 | 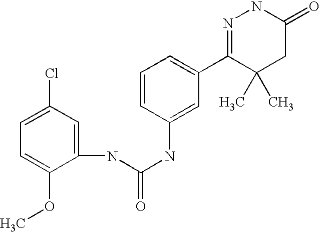 | 401 | 401 | 3.95(93) | 6 |
| 25 | 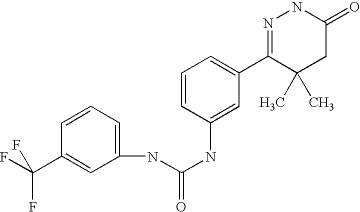 | 404 | 405 | 3.92(100) | 6 |
| 26 | 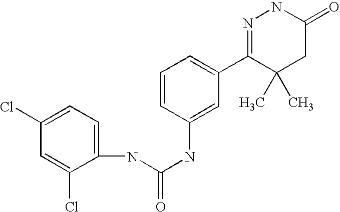 | 405 | 405 | 4.10(100) | 6 |
| 27 | 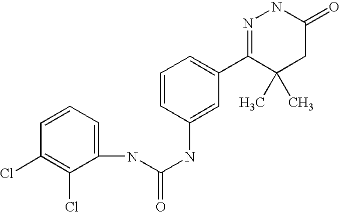 | 405 | 405 | 4.02(100) | 6 |
| 28 | 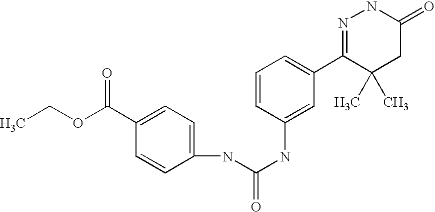 | 408 | 405 | 4.17(82) | 6 |
| 29 | 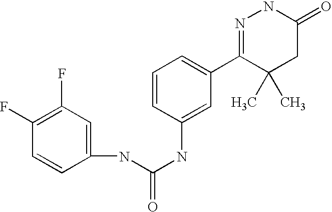 | 372 | 373 | 3.66(100) | 6 |
| 30 | 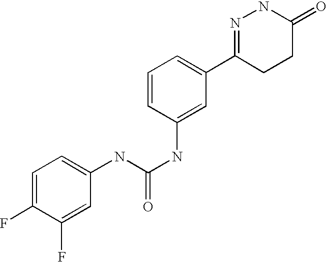 | 344 | 345 | 4.06(92) | 3 |
| 31 | 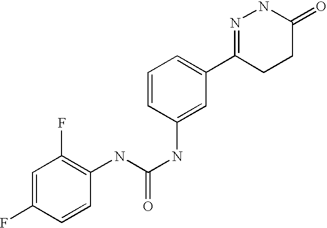 | 344 | 345 | 3.90 | 3 |
| 32 | 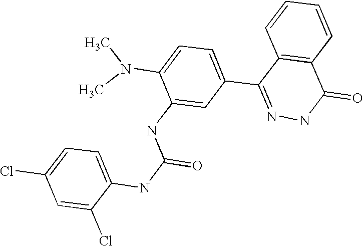 | 468 | 468 | 4.10 | 3 |
| 33 | 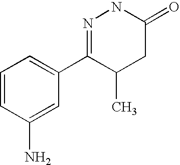 | 203 | MS(EI+) [M]+203 | 2.62 | 3 |
| 34 | 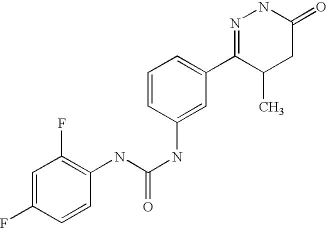 | 358 | MS (DCI/NH3) [M + NH4]+376 | 4.00 | 3 |
| 35 | 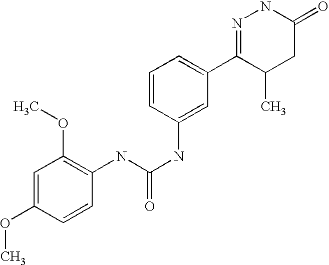 | 382 | 383 | 3.90 | 3 |
| 36 | 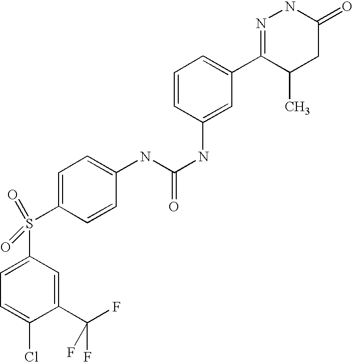 | 565 | 565 | 4.60 | 3 |
| 37 | 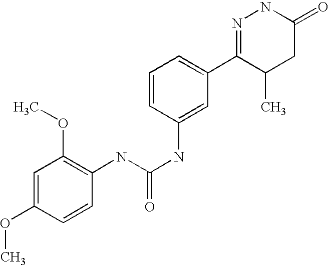 | 382.4 | 383 | 13.64 | 9 |
| 38 | 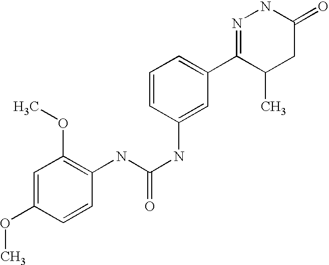 | 382.4 | 383 | 13.97 | 9 |
| 39 | 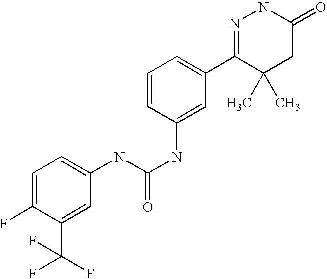 | 422 | 423 | 4.43(100) | 3 |
| 40 | 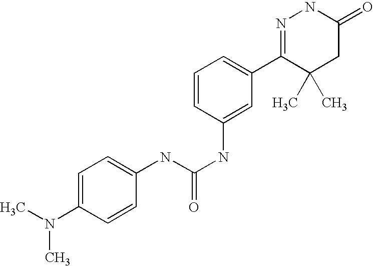 | 379 | 380 | 3.43(100) | 3 |
| 41 | 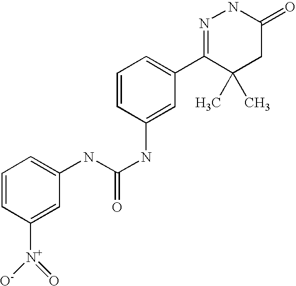 | 381 | 382 | 4.12(100) | 3 |
| 42 | 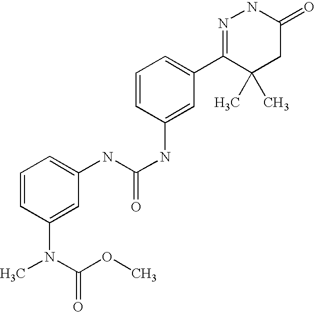 | 423 | 424 | 3.93(100) | 3 |
| 43 | 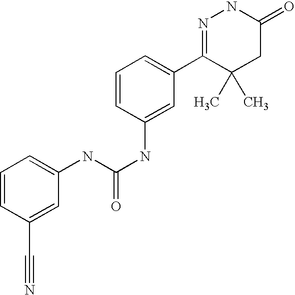 | 361 | 362 | 3.95(100) | 3 |
| 44 | 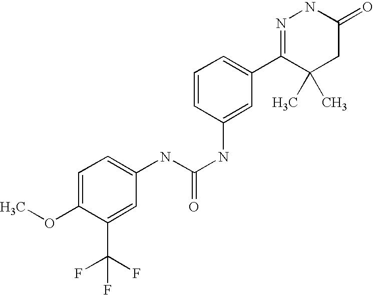 | 434 | 435 | 4.30(100) | 3 |
| 45 | 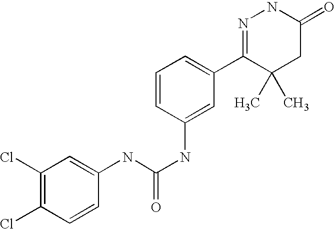 | 405 | 405 | 4.47(100) | 3 |
| 46 | 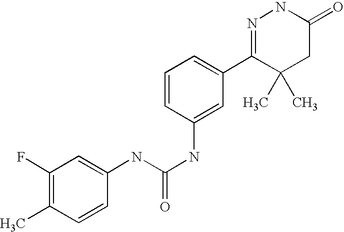 | 368 | 369 | 4.25(100) | 3 |
| 47 | 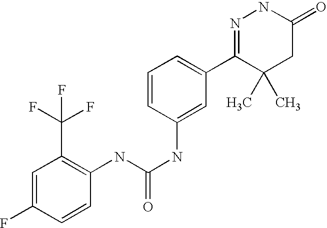 | 422 | 423 | 4.27(100) | 3 |
| 48 | 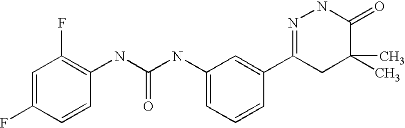 | 372 | 373 | 4.24(100) | 3 |
| 49 | 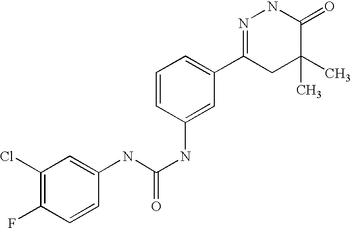 | 389 | 389 | 4.39(100) | 3 |
| 50 | 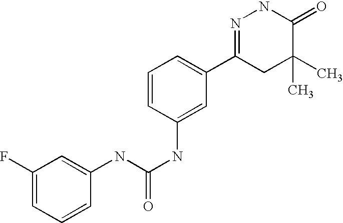 | 354.4 | 355 | 4.24(100) | 3 |
| 51 | 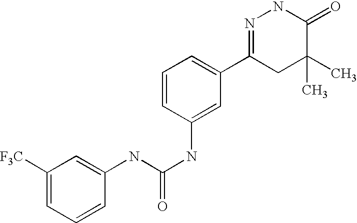 | 404.4 | 405 | 4.47(100) | 3 |
| 52 | 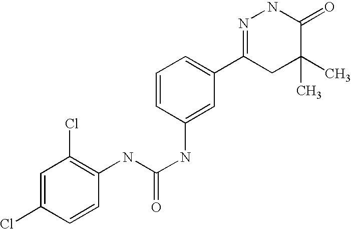 | 405.3 | 405 | 4.64(100) | 3 |
| 53 | 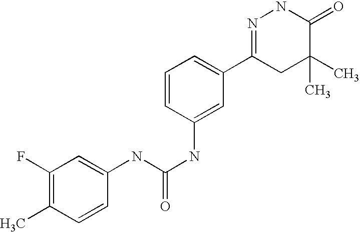 | 368.4 | 369 | 4.36(100) | 3 |
| 54 | 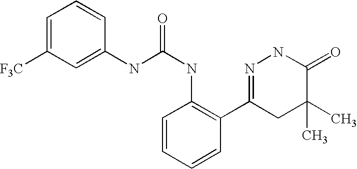 | 404.4 | 405 | 4.84(100) | 3 |
| 55 | 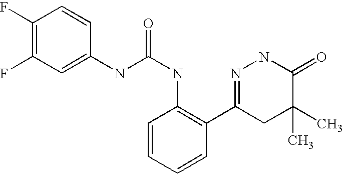 | 372.4 | 373 | 4.62(100) | 3 |
| 56 | 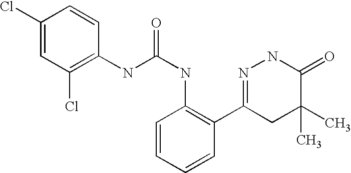 | 405.3 | 405 | 4.76(100) | 3 |
| 57 | 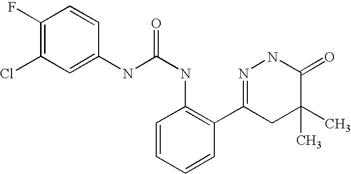 | 388.8 | 389 | 4.64(100) | 3 |
| 58 | 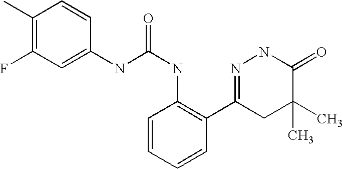 | 368.4 | 369 | 4.54(100) | 3 |
| 59 | 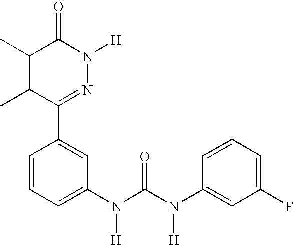 | 354.4 | 355 | 3.64(100) | 6 |
| 60 | 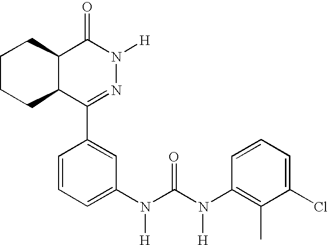 | 410.9 | 412 | 2.87(100) | 8 |

### EXAMPLE 61

### N-(4-Cyano-2-methylphenyl)-N′-[5-(4-ethyl-4-methyl-6-oxo-1,4,5,6-tetrahydro-3-pyridazinyl)-2-methylphenyl]urea

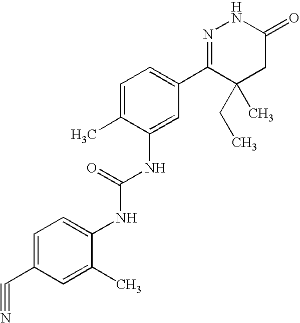

79.3 mg (0.60 mmol) of 4-amino-3-methylbenzonitrile are initially charged in 5 ml of tetrahydrofuran, and 135.66 mg (0.50 mmol) of 5-ethyl-6-(3-isocyanato-4-methylphenyl)-5-methyl-4,5-dihydro-3(2H)-pyridazinone and 1 drop of triethylamine are added. After 20 hours at 50° C., the target compound is filtered off with suction and washed with tetrahydrofuran and diethyl ether. The yield is 28 mg (14%).

¹H-NMR (400 MHz, DMSO): δ=0.73 (t, 3H), 1.15 (s, 3H), 1.44-1.55 (m, 1H), 1.65-1.78 (m, 1H), 2.23 (d, 1H), 2.30 (s, 3H), 2.32 (s, 1H), 2.43 (d, 1H), 7.02 (dd, 1H), 7.20 (dd, 1H), 7.58-7.64 (m, 2H), 7.96 (d, 1H), 8.21 (d, 1H), 8.58-8.62 (m, 2H), 10.91 (s, 1H).

The examples of Table 2 can be obtained according to the general procedure [H].

TABLE 2

| | | | MS(ESI+) | HPLC | |
|---|---|---|---|---|---|
| | | | m/z | Rt[min] | HPLC |
| Example No. | Structure | MW | [M + H]⁺ | (%) | method |
| 62 | 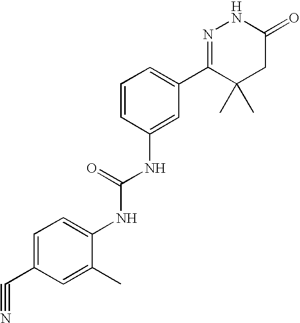 | 375 | 376 | 4.12(100) | 3 |
| 63 | 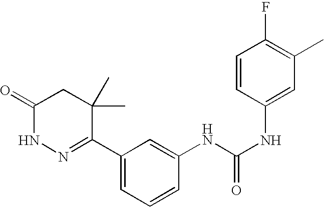 | 368 | 389 | 3.78(98.9) | 5 |
| 64 | 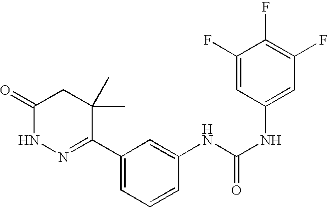 | 390 | 391 | 3.97(100) | 5 |
| 65 | 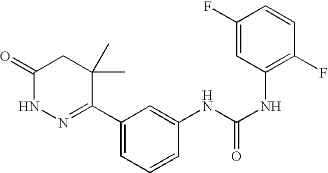 | 372 | 373 | 3.82(98.9) | 5 |
| 66 | 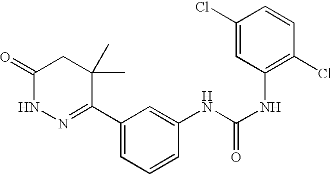 | 404 | 405 | 4.21(100) | 5 |
| 67 | 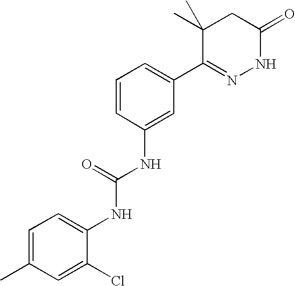 | 384 | 385 | 4.43(100) | 3 |
| 68 | 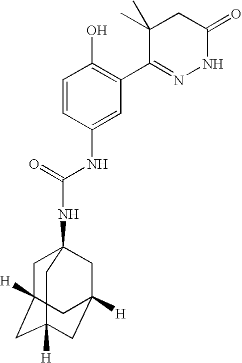 | 410 | 411 | 4.3(100) | 3 |
| 69 | 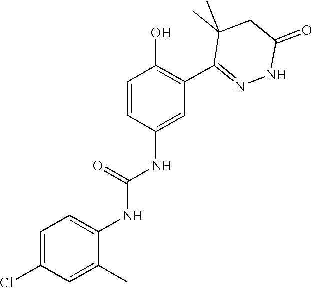 | 400 | 401 | 4.1(100) | 3 |
| 70 | 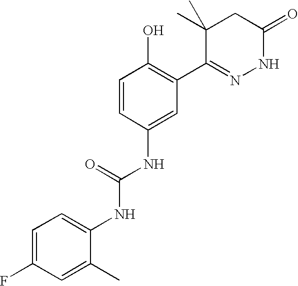 | 384 | 385 | 3.8(96.4) | 3 |
| 71 | 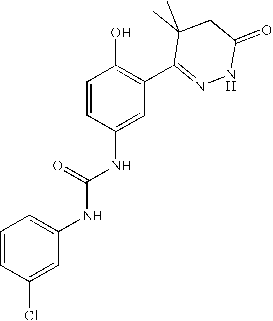 | 386 | 387 | 4.0(96.1) | 3 |
| 72 | 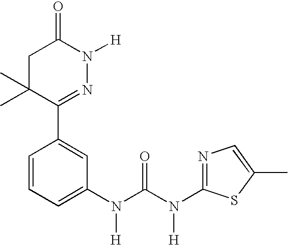 | 357 | 358 | 3.64(94) | 3 |

General LC-MS and HPLC Methods:
HPLC Parameters:

Method 1 (HPLC):

Column: Kromasil C18, L-R

Temperature: 30° C.

Flow rate=0.75 ml/min

Mobile phase=0.01 M HClO₄, B═CH₃CN

Gradient: →0.5 min 98% A→4.5 min 10% A→6.5 min 10% A

Method 2 (HPLC):

Column: Kromasil C18 60*2, L-R

Temperature: 30° C.

Flow rate=0.75 ml/min

Mobile phase=0.01 M H₃PO₄, B═CH₃CN

Gradient: →0.5 min 90% A→4.5 min 10% A→6.5 min 10% A

Method 3 (HPLC):

Column: Kromasil C18 60*2, L-R

Temperature: 30° C.

Flow rate=0.75 ml/min

Mobile phase: A=0.005 M HClO₄, B═CH₃CN

Gradient: →0.5 min 98% A→4.5 min 10% A→6.5 min 10% A

Method 4 (HPLC):

Column: Symmetry C18 2.1×150 mm

Column oven: 50° C.

Flow rate=0.6 ml/min

Mobile phase: A=0.6 g 30% strength HCl/l water, B═CH₃CN

Gradient: 0.0 min 90% A→4.0 min 10% A→9 min 10% A

Method 5 (LC-MS):

MHZ-2Q, Instrument Micromass Quattro LCZ

Column Symmetry C18, 50 mm×2.1 mm, 3.5 μm

Temperature: 40° C.

Flow rate=0.5 ml/min

Mobile phase A=CH₃CN+0.1% formic acid, mobile phase B=water+0.1% formic acid

Gradient: 0.0 min 10% A→4 min 90% A→6 min 90% A

Method 6 (LC-MS):

MHZ-2P, Instrument Micromass Platform LCZ

Column Symmetry C18, 50 mm×2.1 mm, 3.5 μm

Temperature: 40° C.

Flow rate=0.5 ml/min

Mobile phase A=CH₃CN+0.1% formic acid, mobile phase B=water+0.1% formic acid

Gradient: 0.0 min 10% A→4 min 90% A→6 min 90% A

Method 7 (LC-MS):

MHZ-7Q, Instrument Micromass Quattro LCZ

Column Symmetry C18, 50 mm×2.1 mm, 3.5 μm

Temperature: 40° C.

Flow rate=0.5 ml/min

Mobile phase A=CH₃CN+0.1% formic acid, mobile phase B=water+0.1% formic acid

Gradient: 0.0 min 5% A→1 min 5% A→5 min 90% A→6 min 90% A

Method 8 (LC-MS):

Column: Symmetry C 18 2.1×150 mm

Column oven: 50° C.

Flow rate=0.9 ml/min

Mobile phase: A=0.3 g 30% strength HCl/l water, B═CH₃CN

Gradient: 0.0 min 90% A→3.0 min 10% A→6.0 min 10% A

Method 9 (HPLC):

Column: chiral stationary silica gel phase, based on the optically active monomer

N-methacrylacyl-L-leucine-dicyclopropylmethylamide

Flow rate=15 ml/min

Mobile phase: isohexane/ethyl acetate 20:80

Method 10 (HPLC):

Column: Symmetry C18 2.1×150 mm

Column oven: 50° C.

Flow rate=0.9 ml/min

Mobile phase: A=CH₃CN, B=0.3 g 30% strength HCl/l water

Gradient: 0.0 min 10% A→3.0 min 90% A→6.0 min 90% A

Method 11 (HPLC):

Column: Symmetry C18 2.1×150 mm

Column oven: 70° C.

Flow rate=0.9 ml/min

Mobile phase: A=CH₃CN, B=0.3 g 30% strength HCl/l water

Gradient: 0.0 min 2% A→2.5 min 95% A→5.0 min 95% A

Method 12 (preparative HPLC):

Special method developed for removing the para-isomer:

Column: YMC silica gel ODS AQ, 11 μm; 250×30 mm

Flow rate=45 ml/min

Mobile phase: 0.2% strength trifluoroacetic acid/acetonitrile 85/15 (v/v)

Detection: UV @ 220 nm

Sample application: 2.25 ml of a solution of 60 g of product dissolved in 300 ml of

DMSO and 350 ml of 0.2% strength trifluoroacetic acid

Cycle time: 6.1 min

Method WTB (HPLC):

HP1100,

Column: LiChroCart 75-5 LiChrospher 100 RP-18 5 μm

Column oven: 40° C.

Flow rate=2.5 ml/min

Mobile phase: A=water with 0.05% TFA, B═CH₃CN with 0.05% TFA

Gradient: 0.0 min 90% A→0.05 min 90% A→5.0 min 5% A→7.0 min 5% A→7.05 min 90% A→8.0 min 90% A

Preparative HPLC or RP-HPLC:

Reverse Phase

Column: GROM-SIL 120 ODS-4 HE 10 μm, 250*30 mm

Mobile phase: ACN/water gradient

1. A compound of the general formula (I) 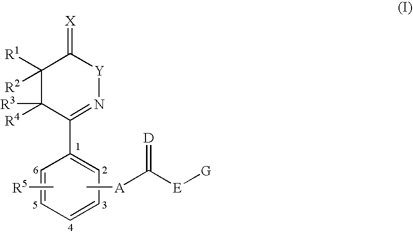
in which
A is attached via position 2, 3, 5 or 6 to the aromatic ring and
A represents oxygen or NR⁶,
E represents oxygen, CR⁹R¹⁰ or NR⁷,
Y represents oxygen or NR⁸,
D and X are identical or different and represent in each case oxygen or sulfur,
G represents hydrogen,
or
G represents C₆-C₁₀-aryl, where C₆-C₁₀-aryl may optionally be substituted by up to three substituents selected from the group consisting of halogen, hydroxyl, nitro, cyano, C₁-C₆-alkoxy, hydroxy-carbonyl, C₁-C₆-alkoxycarbonyl, amino, mono- or di-C₁-C₆-alkylamino, mono- or di-C₁-C₆-alkylaminocarbonyl and C₁-C₆-alkyl,
where
C₁-C₆-alkoxy, C₁-C₆-alkoxycarbonyl, mono- or di-C₁-C₆-alkylamino, mono- or di-C₁-C₆-alkylaminocarbonyl or C₁-C₆-alkyl may optionally be substituted by up to three substituents selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₆-C₁₀-aryl,
or
G represents C₆-C₁₀-aryl, where C₆-C₁₀-aryl may optionally be substituted by phenyl,
where
phenyl may optionally be substituted by up to three substituents selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₁-C₆-alkyl,
where
C₁-C₆-alkyl for its part may optionally be substituted by up to three substituents selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by phenyl,
where
phenyl may optionally be substituted by C₅-C₆-heteroaryl or C₅-C₇-heterocyclyl,
where
C₅-C₆-heteroaryl or C₅-C₇-heterocyclyl for their part may optionally be substituted by up to three substituents selected from the group consisting of halogen, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by a group of the following formula 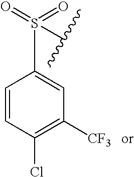
G represents C₅-C₁₀-heteroaryl or C₅-C₇-heterocyclyl, where C₅-C₁₀-heteroaryl or C₅-C₇-heterocyclyl may optionally be substituted by up to three substituents selected from the group consisting of halogen, nitro, cyano, C₁-C₆-alkyl, C₁-C₆-alkoxy, amino, mono- or di-C₁-C₆-alkylamino, hydroxycarbonyl, C₁-C₆-alkoxycarbonyl and mono or di-C₁-C₆-alkylaminocarbonyl,
or
G represents C₃-C₁₀-cycloalkyl, where C₃-C₁₀-cycloalkyl may optionally be substituted by up to three substituents selected from the group consisting of halogen, nitro, cyano, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono or di-C₁-C₆-alkylaminocarbonyl,
R¹, R², R³ and R⁴ are identical or different and each represent hydrogen, amino, mono- or di-C₁-C₆-alkylamino, C₁-C₆-alkylcarbonylamino, C₆-C₁₀-aryl or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono or di-C₁-C₆-alkylaminocarbonyl,
and
where C₆-C₁₀-aryl may optionally be substituted by up to three substituents selected from the group consisting of halogen, hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl, mono or di-C₁-C₆-alkylaminocarbonyl and C₁-C₆-alkyl,
where
C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R² or R³ and R⁴ together with the carbon atom to which they are attached form a C₃-C₆-cycloalkyl ring, where the C₃-C₆-cycloalkyl ring may optionally be substituted by up to three substituents selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R³ together with the carbon atoms to which they are attached form a C₃-C₆-cycloalkyl ring, where the C₃-C₆-cycloalkyl ring may optionally be substituted by up to three substituents selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁵ represents hydrogen, halogen, hydroxyl, C₁-C₆-alkoxy, amino, mono- or di-C₁-C₆-alkylamino or C₁-C₆-alkyl, where C₁-C₆-alkoxy, mono- or di-C₁-C₆-alkylamino or C₁-C₆-alkyl may optionally be substituted by up to three substituents selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁹ and R¹⁰ are identical or different and represent in each case hydrogen, NR¹¹R¹², OR¹³ or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹¹, R¹² and R¹³ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or a tautomer, a stereioisomer, a stereoisomeric mixture or a pharmacologically acceptable salt thereof.

2. A compound of the general formula (I) as claimed in claim 1, in which
A is attached via position 2, 3, 5 or 6 to the aromatic ring, and
A represents oxygen or NR⁶,
E represents oxygen, CR⁹R¹⁰ or NR⁷,
Y represents oxygen or NR⁸,
D and X are identical or different and represent in each case oxygen or sulfur,
G represents hydrogen,
or
G represents C₆-C₁₀-aryl, where C₆-C₁₀-aryl may optionally be substituted by up to three substituents independently of one another selected from the group consisting of halogen, hydroxyl, nitro, cyano, C₁-C₆-alkoxy, hydroxycarbonyl, C₁-C₆-alkoxycarbonyl, amino, mono- or di-C₁-C₆-alkylamino, mono- or di-C₁-C₆-alkylaminocarbonyl and C₁-C₆-alkyl,
where
C₁-C₆-alkoxy, C₁-C₆-alkoxycarbonyl, mono- or di-C₁-C₆-alkylamino, mono- or di-C₁-C₆-alkylaminocarbonyl or C₁-C₆-alkyl may optionally be substituted by up to three substituents independently of one another selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₆-C₁₀-aryl,
or
G represents C₆-C₁₀-aryl, where C₆-C₁₀-aryl may optionally be substituted by phenyl,
where
phenyl may optionally be substituted by up to three substituents independently of one another selected from the group consisting of halogen, hydroxyl, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₁-C₆-alkyl,
where
C₁-C₆-alkyl for its part may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by phenyl,
where
phenyl may optionally be substituted by C₅-C₆-heteroaryl or C₅-C₇-heterocyclyl,
where
C₅-C₆-heteroaryl or C₅-C₇-heterocyclyl for their part may optionally be substituted by up to three substituents independently of one another selected from the group consisting of halogen, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₆-C₁₀-aryl, where C₆-C₁₀-aryl may optionally be substituted by a group of the following formula 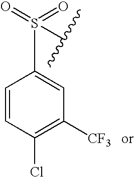
G represents C₅-C₁₀-heteroaryl or C₅-C₇-heterocyclyl, where C₅-C₁₀-heteroaryl or C₅-C₇-heterocyclyl may optionally be substituted by up to three substituents independently of one another selected from the group consisting of halogen, nitro, cyano, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G represents C₃-C₁₀-cycloalkyl, where C₃-C₁₀-cycloalkyl may optionally be substituted by up to three substituents independently of one another selected from the group consisting of halogen, nitro, cyano, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹, R², R³ and R⁴ are identical or different and represent in each case hydrogen, amino, mono- or di-C₁-C₆-alkylamino, C₁-C₆-alkylcarbonylamino, C₆-C₁₀-aryl or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
and
where C₆-C₁₀-aryl may optionally be substituted by up to three substituents selected from the group consisting of halogen, hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl, mono- or di-C₁-C₆-alkylaminocarbonyl and C₁-C₆-alkyl,
where
C₁-C₆-alkyl may optionally be substituted by up to three substituents independently of one another selected from the group consisting of hydroxyl, C₁-C₆-alkoxy, amino,-mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
where R¹, R², R³ and R⁴ are not simultaneously hydrogen,
or
R¹ and R² or R³ and R⁴ together with the carbon atom to which they are attached form a C₃-C₆-cycloalkyl ring, where the C₃-C₆-cycloalkyl ring may optionally be substituted by up to three substituents independently of one another selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R³ together with the carbon atoms to which they are attached form a C₃-C₆-cycloalkyl ring, where the C₃-C₆-cycloalkyl ring may optionally be substituted by up to three substituents independently of one another selected from the group consisting of halogen, hydroxyl, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁵ represents hydrogen, halogen, hydroxyl, C₁-C₆-alkoxy, amino, mono- or di-C₁-C₆-alkylamino or C₁-C₆-alkyl, where C₁-C₆-alkoxy, mono- or di-C₁-C₆-alkylamino or C₁-C₆-alkyl may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are identical or different and represent in each case hydrogen or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁹ and R¹⁰ are identical or different and represent in each case hydrogen, NR¹¹R¹², OR¹³ or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹¹, R¹² and R¹³ are identical or different and represent in each case hydrogen or C₁-C₆-alkyl, where C₁-C₆-alkyl may optionally be substituted by up to three substituents selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or a tautomer, a stereoisomer, a stereoisomeric mixture or a pharmacologically acceptable salt thereof.

3. A compound of the general formula (I) as claimed in claim 1 or 2, in which
A is attached via position 2, 3, 5 or 6 to the aromatic ring and
A represents NR⁶,
E represents NR⁷,
Y represents NR⁸,
D and X represent oxygen,
G represents C₆-C₁₀-aryl, where C₆-C₁₀-aryl may optionally be substituted by up to three substituents independently of one another selected from the group consisting of halogen, hydroxyl, cyano and C₁-C₆-alkyl,
where
C₁-C₆-alkyl may optionally be substituted by up to three substituents of halogen,
or
G represents C₅-C₆-heteroaryl, where C₅-C₆-heteroaryl may optionally be substituted by up to three substituents independently of one another selected from the group consisting of halogen and C₁-C₃-alkyl,
or
G represents C₃-C₁₀-cycloalkyl, where C₃-C₁₀-cycloalkyl may optionally be substituted by up to three substituents C₁-C₆-alkyl,
R¹, R² and R³ are identical or different and represent in each case hydrogen or represent C₁-C₃-alkyl,
R⁴ represents hydrogen, C₆-C₁₀-aryl or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
and
where C₆-C₁₀-aryl may optionally be substituted by up to three substituents independently of one another selected from the group consisting of halogen, hydroxyl, C₁-C₆-alkoxy and C₁-C₆-alkyl,
where R¹, R², R³ and R⁴ do not simultaneously represent hydrogen,
R⁵ represents hydrogen, halogen, hydroxyl, amino, mono- or di-C₁-C₆-alkylamino or C₁-C₆-alkyl, where C₁-C₆-alkyl may optionally be substituted by up to three substituents independently of one another selected from the group consisting of hydroxyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ represent hydrogen,
or a tautomer, a stereoisomer, a stereoisomeric mixture or a pharmacologically acceptable salt thereof.

4. A compound of the general formula (I) as claimed in claim 1, 2 or 3, where the radical A is attached via position 3 to the aromatic ring.

5. A compound of the general formula (I) as claimed in claim 1, 2 or 3, where D and X and represent oxygen.

6. A compound of the general formula (I) as claimed in claim 1, 2 or 3, where A, E and Y represent NH.

7. A compound of the general formula (I) as claimed in claim 1, 2 or 3, where G represents substituted phenyl.

8. A compound of the general formula (I) as claimed in claim 1, 2 or 3, where R¹, R² and R⁵ represent hydrogen and R³ and R⁴ represent methyl.

9. A process for preparing the compounds of the formula (I) as claimed in claim 1, which comprises
[A] reacting compounds of the general formula (II) 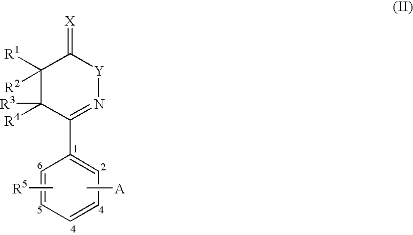
in which
A is attached via position 2, 3, 5 or 6 to the aromatic ring and
R¹, R², R³, R⁴, R⁵, A, X and Y are as defined above,
with compounds of the general formula (III)
D=C═N-G (III)
in which
D and G are as defined above
to give compounds of the general formula (Ia) 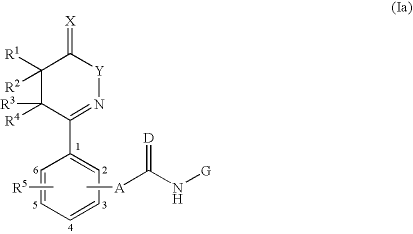
in which
A is attached via position 2, 3, 5 or 6 to the aromatic ring and
R¹, R², R³, R⁴, R⁵, A, D, G, X and Y are as defined above, or
[B] reacting compounds of the general formula (II) with compounds of the general formula (IV) 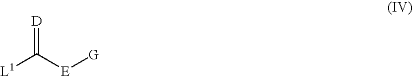
in which
D, E and G are as defined above and
L¹ represents p-nitrophenyl or halogen, preferably bromine or chlorine,
to give compounds of the general formula (I) 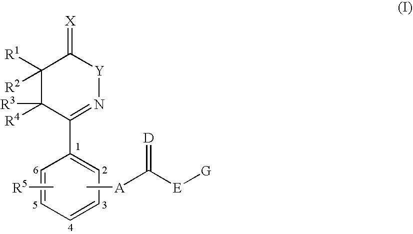
in which
A is attached via position 2, 3, 5 or 6 to the aromatic ring and
R¹, R², R³, R⁴, R⁵, A, D, E, G, X and Y are as defined above, or
[C] reacting compounds of the general formula (V) 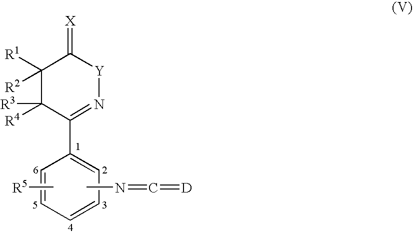
in which
—NCD is attached via position 2, 3, 5 or 6 to the aromatic ring and
R¹, R², R³, R⁴, R⁵, D, X and Y are as defined above
with compounds of the general formula (VI)
H-M-G (VI)
in which
G is as defined above and
M represents oxygen or NR⁷,
where
R⁷ is as defined above,
to give compounds of the general formula (Ib) 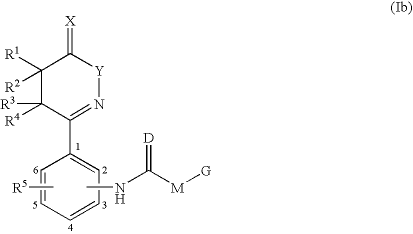
in which
—NH—C(D)-M-G is attached via position 2, 3, 5 or 6 to the aromatic ring and
R¹, R², R³, R⁴, R⁵, D, G, M, X and Y are as defined above.

10. A compound of the general formula (I) as claimed in claim 1, 2 or 3 for controlling disorders.

11. A medicament, comprising compounds of the general formula (I) as claimed in claim 1, 2 or 3 in combination with at least one pharmaceutically acceptable, pharmaceutically safe carrier or excipient.

12. The use of compounds of the general formula (I) as claimed in claim 1, 2 or 3 for preparing as medicament for treating viral disorders.

13. A medicament as claimed in claim 12, for treating viral disorders.

14. A method for controlling viral disorders in humans and animals by administration of an antivirally effective amount of at least one compound as claimed in any of claims 1 to 3.
